SEQUENCE LISTING (10 sequences)

SEQ ID NO: 1 - DNA, 378 bp, Homo sapiens
atgccgtcggagaagaccttcaagcagcgccgcaccttcgaacaaagagtagaagatgtccgacttattcgagagcagcatccaaccaaaatcccggtgataatagaacgatacaagggtgagaagcagcttcctgttctggataaaacaaagttccttgtacctgaccatgtcaacatgagtgagctcatcaagataattagaaggcgcttacagctcaatgctaatcaggccttcttcctgttggtgaacggacacagcatggtcagcgtctccacaccaatctcagaggtgtatgagagtgagaaagatgaagatggattcctgtacatggtctatgcctcccaggagacgttcgggatgaaattgtcagtgtaa

SEQ ID NO: 2 - DNA, 1161 bp, Homo sapiens
atgagtcttctaaccgaggtcgaaacgtacgttctctctatcgtcccgtcaggccccctcaaagccgagatcgcacagagacttgaagatgtctttgcagggaagaacaccgatcttgaggttctcatggaatggctaaagacaagaccaatcctgtcacctctgactaaggggattttaggatttgtgttcacgctcaccgtgcccagtgagcggggactgcagcgtagacgctttgtccaaaatgctcttaatgggaacggagatccaaataacatggacaaagcagttaaactgtataggaagcttaagagggagataacattccatggggccaaagaaatagcactcagttattctgctggtgcacttgccagttgtatgggcctcatatacaacaggatgggggctgtgaccactgaagtggcatttggcctggtatgcgcaacctgtgaacagattgctgactcccagcatcggtctcataggcaaatggtgacagcaaccaatccactaatcagacatgagaacagaatggttctagccagcactacagctaaggctatggagcaaatggctggatcgagtgagcaagcagcagaggccatggatattgctagtcaggccaggcaaatggtgcaggcgatgagaaccattgggactcatcctagctccagtgctggtctaaaagatgatcttcttgaaaatttgcaggcctatcagaaacgaatgggggtgcagatgcaacgattcaaggactcgagctcaagcttcgaattcaccatgccgtcggagaagaccttcaagcagcgccgcaccttcgaacaaagagtagaagatgtccgacttattcgagagcagcatccaaccaaaatcccggtgataatagaacgatacaagggtgagaagcagcttcctgttctggataaaacaaagttccttgtacctgaccatgtcaacatgagtgagctcatcaagataattagaaggcgcttacagctcaatgctaatcaggccttcttcctgttggtgaacggacacagcatggtcagcgtctccacaccaatctcagaggtgtatgagagtgagaaagatgaagatggattcctgtacatggtctatgcctcccaggagacgttcgggatgaaattgtcagtgtaa

SEQ ID NO: 3 - protein, 386 aa, Homo sapiens
MSLLTEVETYVLSIVPSGPLKAEIAQRLEDVFAGKNTDLEVLMEWLKTRPILSPLTKGILGFVFTLTVPSERGLQRRRFVQNALNGNGDPNNMDKAVKLYRKLKREITFHGAKEIALSYSAGALASCMGLIYNRMGAVTTEVAFGLVCATCEQIADSQHRSHRQMVTATNPLIRHENRMVLASTTAKAMEQMAGSSEQAAEAMDIASQARQMVQAMRTIGTHPSSSAGLKDDLLENLQAYQKRMGVQMQRFKDSSSSFEFTMPSEKTFKQRRTFEQRVEDVRLIREQHPTKIPVIIERYKGEKQLPVLDKTKFLVPDHVNMSELIKIIRRRLQLNANQAFFLLVNGHSMVSVSTPISEVYESEKDEDGFLYMVYASQETFGMKLSV

SEQ ID NO: 4 - protein, 16 aa, Homo sapiens
MPSEKTFKQRRTFEQR

SEQ ID NO: 5 - DNA, 22 bp, Homo sapiens
ucaacuugcuacuacaugaudt

SEQ ID NO: 6 - DNA, 22 bp, Homo sapiens
ucauguaguagcaaguugaudt

SEQ ID NO: 7 - DNA, 23 bp, Homo sapiens
cuggacuuccagaagaacadtdt

SEQ ID NO: 8 - DNA, 23 bp, Homo sapiens
uguucuucuggaaguccagdtdt

SEQ ID NO: 9 - DNA, 23 bp, Photuris pennsylvanica
cguacgcggaauacuucgadtdt

SEQ ID NO: 10 - DNA, 23 bp, Photuris pennsylvanica
ucgaaguauuccgcguacgdtdt

(19) United States
(12) Patent Application Publication
(10) Pub. No.: US 2010/0266541 A1
(43) Pub. Date: Oct. 21, 2010

(54) METHODS AND AGENTS FOR MODULATING AN IMMUNE RESPONSE

(76) Inventor: Christian Munz, New York, NY (US)

Correspondence Address: FOX ROTHSCHILD LLP;PRINCETON PIKE CORPORATE CENTER, 997 LENOX DRIVE, BLDG. #3, LAWRENCEVILLE, NJ, 08648, US

(21) Appl. No.: 12/280,269

(22) PCT Filed: Feb. 20, 2007
(86) PCT No.: PCT/US07/04208

§ 371 Date: Aug. 21, 2008

Related U.S. Application Data
(60) Provisional application No. 60/774,614, filed on Feb. 21, 2006.

(51) Int. Cl. A61K 38/21; A61K 39/00; A61K 39/12; A61K 39/04; A61K 39/02; A61K 35/12; C12N 5/10; C12N 5/02; C12N 15/00; C07H 21/04; A61P 37/00

(52) U.S. Cl. 424/85.5; 424/184.1; 424/204.1; 424/234.1; 424/248.1; 424/93.71; 435/325; 435/375; 435/320.1; 536/23.1; 536/23.72

(57) ABSTRACT

This invention is directed to the targeting of proteins or peptides of interest to an autophagosome, for their presentation on a major histocompatibility complex (MHC) class II molecule and methods of use thereof. Nucleic acids, vectors comprising the same, and compositions for targeting proteins or peptides of interest to an autophagosome, for their presentation on a major histocompatibility complex (MHC) class II molecule are disclosed as are methods of use thereof for stimulating or enhancing immune responses in a subject.

### CROSS-REFERENCE TO RELATED APPLICATION

This application claims priority to U.S. provisional patent application Ser. No. 60/774,614, filed Feb. 21, 2006, herein incorporated by reference in its entirety.

### STATEMENT AS TO FEDERALLY SPONSORED RESEARCH

This invention was conducted with U.S. Government support under National Institutes of Health grant no. CA108609. The government has certain rights in the invention.

### BACKGROUND OF THE INVENTION

The T cells of the adaptive immune system monitor all body cells for the presence of pathogenic proteins. For this purpose, peptides generated by the proteasome are presented on MHC class I molecules and recognized by CD8+ T cells, whereas products of lysosomal degradation are presented on MHC class II molecules and recognized by CD4+ T cells. Antigens presented on MHC class II typically are exogenous proteins that are endocytosed by the antigen presenting cell (APC) or endogenous proteins that reside in the secretory system. However, analysis of peptides eluted from is MHC class II molecules has revealed that a significant proportion of natural MHC class II ligands (up to 20%) are derived from cytosolic and nuclear proteins. Furthermore, it was shown that CD4+ T cells can recognize cytosolic and nuclear antigens after endogenous processing, for example, the fact that cytosolic measles virus and influenza A virus antigens can be endogenously processed for MHC class II presentation. Subsequently, endogenous MHC class II presentation has been described for other viral antigens, self antigens, model antigens, as well as tumor antigens. Therefore, antigens that are topologically isolated from the endosomal system can gain access to the MHC class II antigen presentation pathway and broaden the repertoire of MHC class II ligands.

Different processing pathways have been discussed to contribute to endogenous MHC class II antigen presentation. Recently, autophagy was shown to deliver cytosolic and nuclear antigens onto MHC class. II molecules. Of these, physiological levels of the Epstein-Barr virus (EBV) nuclear antigen 1 (EBNA1), as the first pathogen-derived antigen, were found to be presented on MHC class II molecules of EBV-transformed B cell lines after macroautophagic degradation.

Autophagy consists of at least three intracellular protein degradation pathways found ubiquitously in eukaryotic cells, macroautophagy, microautophagy and chaperone mediated autophagy. Most evidence for endogenous MHC class II antigen processing has implicated the macroautophagy pathway. During macroautophagic degradation, cytoplasmic material including organelles are sequestered into double-membrane coated autophagosomes, which subsequently fuse with endosomes and lysosomes. The sequestered contents of autophagosomes are then broken down by lysosomal hydrolases and the degradation products are recycled by the cell.

Manipulation of this pathway would, in theory, pose a means of accessing the MHC class II presentation pathway as a means of promoting immune responses, though as yet such a means is unknown.

### SUMMARY OF THE INVENTION

In one embodiment, a nucleic acid is provided encoding a peptide or protein of interest fused in frame to a nucleic acid encoding the autophagosomal LC3 protein, or a functional fragment thereof, wherein said peptide or protein of interest is poorly or not presented efficiently on a major histocompatibility complex (MHC) class II molecule.

In another embodiment, the nucleic acid has a sequence homologous to, or corresponding to SEQ ID NO: 1. In a further embodiment, the peptide or protein of interest is virally encoded, and in one embodiment, the peptide or protein of interest is encoded by the influenza virus, which in another embodiment, is a matrix protein, and in another embodiment, the nucleic acid has a sequence homologous to, or corresponding to SEQ ID NO: 2.

In another embodiment, a vector or cell is provided comprising the nucleic acids described herein. In another embodiment, a cell is provided comprising the vectors described herein.

In one embodiment, a method is provided for stimulating or enhancing presentation of a peptide or protein of interest in the context of a major histocompatibility (MHC) class II molecule, the method comprising contacting a cell capable of expressing a major histocompatibility complex (MHC) class II molecule with a nucleic acid encoding a peptide or protein of interest fused in frame to a nucleic acid encoding an autophagosomal targeting protein, or a functional fragment thereof, whereby said autophagosomal targeting protein or a functional fragment thereof targets said peptide or protein of interest to an autophagosome, and said peptide, or a fragment of said protein of interest is displayed on the surface of said cell in the context of a major histocompatibility complex (MHC) class II molecule.

In one embodiment, the autophagosomal targeting protein is an LC3 protein. In one embodiment, the nucleic acid comprises a sequence homologous to, or corresponding to SEQ ID NO: 1. In one embodiment, the cell is diseased. In another embodiment, the cell is infected, and in one embodiment, the cell is infected with a virus, which, in one embodiment is influenza or, in another embodiment, HIV. According to this aspect, and in one embodiment, the peptide or protein of interest is virally encoded, in one embodiment, by a vaccinia virus or a lentivirus. In one embodiment, the peptide or protein of interest is a matrix protein, and in one embodiment, the nucleic acid according to this aspect, has a sequence homologous to, or corresponding to SEQ ID NO: 2.

In one embodiment, the cell is infected with a bacterium, which in one embodiment, is a mycobacterium. In another embodiment, the cell is neoplastic or preneoplastic.

In one embodiment, the cell is contacted with a cytokine, which in one embodiment, is interferon-γ.

In one embodiment, a method is provided for stimulating or enhancing an immune response in a subject, the method comprising contacting a cell capable of expressing a major histocompatibility complex (MHC) class II molecule in said subject with a nucleic acid encoding a peptide or protein of interest fused in frame to a nucleic acid encoding an autophagosomal targeting protein, or a functional fragment thereof, whereby said autophagosomal targeting protein or functional fragment thereof targets said peptide or protein of interest to an autophagosome, and said peptide, or a fragment of said protein of interest is displayed on the surface of said cell in the context of a major histocompatibility complex (MHC) class II molecule.

According to this aspect, and in one embodiment, the cell is contacted indirectly with the nucleic acid or vector comprising the same. In one embodiment, the nucleic acid or vector comprising the same is administered intravenously to the subject, and in another embodiment, the subject is administered a composition comprising said nucleic acid or vector comprising the same. In one embodiment, the composition is administered repeatedly, over a course of time. In one embodiment, the composition comprises a neoplastic cell isolated from the subject, which in one embodiment, is contacted ex vivo with the nucleic acid or vector comprising the same. In one embodiment, the subject has preneoplastic or hyperplastic cells or tissue, or in another embodiment, the subject is predisposed to neoplasia.

In one embodiment, a composition is provided comprising a cell capable of expressing the major histocompatibility complex (MHC) class II protein, and a nucleic acid encoding a peptide or protein of interest fused in frame to a nucleic acid encoding the autophagosomal LC3 protein, or functional fragment thereof, or a vector comprising the same.

In another embodiment, a method is provided for downmodulating, suppressing or tolerizing an immune response in a subject to a peptide or protein of interest, the method comprising contacting immature dendritic cells with a nucleic acid encoding a peptide or protein of interest fused in frame to a nucleic acid encoding an autophagosomal targeting protein, or a functional fragment thereof, whereby said autophagosomal targeting protein or functional fragment thereof targets said peptide or protein of interest to an autophagosome, and said peptide, or a fragment of said protein of interest is displayed on the surface of said immature dendritic cell in the context of a major histocompatibility complex (MHC) class II molecule. In one embodiment, the cell is contacted in vivo or ex vivo with a vector comprising the nucleic acid. In another embodiment, the autophagosomal targeting protein is an LC3 protein. In yet another embodiment, the nucleic acid comprises a sequence homologous to, or corresponding to SEQ ID NO: 1.

According to this aspect, and in one embodiment, the downmodulating, suppressing or tolerizing an immune response is to prevent or diminish transplant rejection in the subject. In another embodiment, the peptide or protein of interest is a graft antigen or a host antigen. In yet another embodiment, the downmodulating, suppressing or tolerizing an immune response is to treat autoimmunity in the subject. In another embodiment, the peptide or protein of interest is a self antigen.

### BRIEF DESCRIPTION OF THE DRAWINGS

FIGS. 1 A-G demonstrate autophagosome formation in human epithelial cell lines. (a) Constitutive autophagosome formation in human epithelial cell lines. Human epithelial cell lines HaCat (keratinocyte), HeLa (cervical carcinoma), MDAMC (breast carcinoma) and 293 (kidney) were stably transfected with the GFP-LC3 reporter construct. To stabilize GFP-LC3 in endosomal/lysosomal compartments, cells were treated with 50 μM chloroquine for 10 h (+CQ, right column). Cells were fixed, stained with DAPI and analyzed by confocal microscopy. Scale bars: 20 μm. Representative fields from one experiment out of three are shown. (b) Macroautophagy is a constitutive process in professional antigen-presenting cells, including dendritic cells. To assess lysosomal turnover of GFP-LC3 in professional APCs, an EBV-transformed B lymphocyte cell line (B-LCL) stably expressing GFP-LC3 and GFP-LC3-expressing immature and mature DCs (iDC and mDC) were treated with 50 mM chloroquine for 10 hr (+CQ) or were left untreated (no CQ). Cells were tixed, stained with DAPI, and analyzed by confocal microscopy. Scale bars represent 20 mm. Representative fields from one experiment out of two are shown. (c) GFP-LC3 is degraded in MHC class II-loading compartments of dendritic cells. Upper panel: GFP-LC3-expressing immature DCs were treated with 50 mM chloroquine for 10 hr (+CQ). Cells were stained with an MHC class II-specific antibody, and DAPI and colocalization of GFP-LC3 with MHC class II was analyzed by confocal microscopy. Scale bar represents 10 um. Representative cells from one experiment out of three are shown. Lower panel: Same experiment as in upper panel was performed with mature DCs. In the majority of mature DCs, MHC class II was mainly localized at the cell surface, but a subset of cells had intracellular MHC class II compartments (white arrow). Scale bar represents 10 μm. Representative cells from one experiment out of three are shown. (d) Macroautophagy is required for delivery of MP1-LC3 to MHC class II-loading compartments. MDAMC cells stably expressing MP1-LC3 were transfected with control siRNA (specific for firefly luciferase) or siRNA specific for atg12. After 36 h, cells were treated with 200 U/ml IFNγ to upregulate MHC class II expression and were cultured for another 36 h. To prevent degradation of MP1-LC3 by lysosomal proteases, cells were treated with 50 μM chloroquine (CQ) during the last 6 hours of the culture, where indicated (+CQ). Cells were fixed, stained with MP1- and MHC class II specific antibodies and DAPI and analyzed by confocal microscopy. Scale bar: 10 μm. Representative fields from one experiment out of two are shown. In control siRNA-treated cells, a substantial fraction of MP1-LC3-containing vesicles can be observed to colocalize with MHC class II compartments, whereas this colocalization is completely abrogated after atg12. (e-f) Chloroquine treatment induces gradual accumulation of GFP-LC3 in autolysosomes and differs substantially from nutrient starvation. (e) 293 cells stably transfected with a GFP-LC3 reporter construct were left untreated (no CQ) or were treated with 50 μM chloroquine (CQ) for 2 or 10 hours. Cells were fixed, stained with DAPI and analyzed by fluorescence microscopy. Inhibition of lysosomal acidification with CQ leads to a gradual accumulation of GFP-LC3-labeled autophagosomes over time. Representative fields from one experiment out of two are shown. (f) MDAMC cells were left untreated (--), cultured in Hanks Balanced Salt Solution (starv.), treated with 50 μM chloroquine (+CQ) or with the protease inhibitors E64 (28 μM), Leupeptin (40 μM) and Pepstatin A (15 μM) (+Prot. inhib.) for 10 hours. Whole cell lysates were run on a 12% SDS-PAGE gel and LC3-I and II were visualized by anti-LC3 Western blotting. Actin blot demonstrates equal protein loading. While nutrient starvation induces LC3-II only weakly, inhibition of lysosomal proteases by treatment with CQ or the protease inhibitors E64, Leupeptin and Pepstatin A leads to a strong accumulation of LC3-II. One of two experiments is shown. (g) MDAMC cells stably expressing GFP-LC3 were either mock transfected or transfected with siRNA duplexes specific for lamin A/C or ATG12. After 2 days, cells were treated with 50 mM CQ for 6 hr (+CQ) or were left untreated (no CQ), stained with DAPI, and examined in an epifluorescence microscope. One of two experiments is shown.

FIGS. 2 A-C demonstrate constitutive autophagy in human epithelial cell lines and professional APCs, including primary monocytes/dendritic cells. (a) Human epithelial cell lines [HaCat (keratinocyte), HeLa (cervical carcinoma), MDAMC (breast carcinoma) and 293 (kidney)] and B cell lines [MS-LCL (EBV-transformed B lymphoblastoid cell line), RPMI6666 and L591 (Hodgkin's lymphoma cell lines)] and (b) primary CD14+ monocytes and monocyte-derived dendritic cells, immature or matured with LPS, were treated for 10 h with 50 μM chloroquine (+) or were left untreated (--). Whole cell lysates were run on 12% SDS-PAGE gels and LC3-I and -II were visualized by anti-LC3 Western blotting. Actin blots show that CQ-treatment did not affect general protein levels. One of three experiments is shown (c) The HaCat keratinocyte cell line was treated with 50 μM chloroquine for 0, 1, 2, 10 or 24 h and accumulation of LC3-II was analyzed by anti-LC3 Western blot. Actin blots controls for sample loading. One of two experiments is shown.

FIGS. 3 A-D demonstrate the autophagosome marker GFP-LC3 colocalizes with markers of MHC class II loading compartments. (a) Upregulation of surface MHC class II after treatment of human epithelial cell lines with 200 U/ml IFNγ for 48 h. Cells were stained with an anti-MHC class II antibody and analyzed by flow cytometry. One of two experiments is shown. (b) The MDAMC breast carcinoma cell line was treated with 200 U/ml IFNγ, transiently transfected with the pEGFP-LC3 reporter construct and 36 h later stained with antibodies to MHC class II, HLA-DM, LAMP-2 and DAPI for DNA content. Colocalization of GFP-LC3 with MIIC markers was analyzed by confocal microscopy. (c) Same as (b), except that 50 μM chloroquine were present during the last 10 h of the culture. Scale bars: 10 μm. Representative cells from one experiment out of three are shown. (d) Recombinant IFNs do not affect macroautophagy in human epithelial cells. Human epithelial cell lines (293T, HaCat and MDAMC) were treated for 24 h with 1000 U/ml recombinant human IFN-a or IFN-γ or were left untreated (--). Whole cell lysates were prepared and equal amounts of protein were run on a 12% SDS-PAGE gel. LC3-I and -II were visualized by anti-LC3 Western blotting. The high molecular weight bands marked with an asterisk (*) are proteins that cross-react with the LC3 antiserum and demonstrate equal protein loading. LC3-II levels and hence macroautophagy are not affected by the IFN treatment.

FIGS. 4 A-C demonstrate the autophagosome marker GFP-LC3 does not colocalize with markers of early/recycling endosomes or MHC class I loading compartments. The MDAMC breast carcinoma cell line was transiently transfected with the pEGFP-LC3 reporter construct and 24 h later treated with 50 μM CQ for 10 h. Cells were stained with antibodies to (a) early endosomal antigen (EEA1) or transferrin receptor (TR) and (b) MHC class I. In addition, cells were stained with DAPI for DNA content and analyzed by confocal microscopy. Scale bars: 10 μm. Representative cells from one experiment out of two are shown. (c) Quantitative analysis for colocalization of GFP-LC3 with MHC class II, HLA-DM, and EEA1 in untreated or CQ-treated MDAMC cells. Data represent means from 10-15 cells from one representative experiment out of two. Error bars indicate standard deviations. p values from homocedastic, one-tailed Student's t test statistics are shown.

FIGS. 5 A-F demonstrate the colocalization of GFP-LC3 and MHC class II molecules in electron-dense multivesicular compartments. (a-d) Untreated (a, b) or CQ-treated (c, d) MDAMC epithelial cells stably expressing GFP-LC3 and MHC class II positive due to IFNγ induction were fixed in 4% paraformaldehyde and cut into 80 nm-thin cryosections. Sections were labeled with an HLA-DR-specific antiserum and 10 nm protein A-Gold (PAG10) and antibody-PAG complexes were irreversibly fixed with glutaraldehyde. Subsequently, sections were labeled with a GFP-specific antibody and 15 nm protein A-Gold (PAG15) and were analyzed by electron microscopy. As a control, PAG10 and PAG15 were interchanged and were shown to produce the same labeling pattern (a, b vs. c, d). Insets from panels a and c are shown magnified in panels b and d, respectively. Representative fields from one experiment out of three are shown. (e-f) Immunoelectron microscopy of MHC class IUGFP-LC3-labeled cryosections (e) Ultrathin cryosections of PFA-fixed MDAMC-GFP-LC3 cells were double-labeled with anti-HLA-DR antiserum/15 nm gold particles and anti-GFP antiserum/10 nm gold particles and analyzed by electron microscopy. MHC class II labeling can be seen both on GFP-LC3-positive electron-dense multivesicular compartments and on the plasma membrane. One representative field from one experiment out of three is shown. Scale bar: 1 μm. (f) MDAMC-GFP-LC3 cells were treated with 50 μM CQ for 10 h and ultrathin crysections were double-labeled for MHC class II (10 nm gold) and GFP (15 nm gold) and analyzed by electronmicroscopy. Double-labeled multivesicular compartments frequently appear expanded and swollen, with a diameter of >1 μm and some empty space. Three representative fields from one experiment out of three are shown. Scale bar: 1 μm.

FIG. 6 A-D demonstrate the targeting of influenza A matrix protein 1 to autophagosomes by fusion to Atg8/LC3. The influenza A matrix protein 1 (MP1) coding sequence was fused to the N-terminus of the LC3 sequence, either with or without a stop codon at the 3′ end of MP1. (a) Schematic diagram of the two constructs encoding for MP1 and MP1-LC3, respectively. (b) HaCat and MDAMC cell lines were stably transfected with MP1 and MP1-LC3 lentiviral constructs and protein expression was analyzed by Western blot with anti-MP1 antiserum. Actin blot shows equal protein loading. (c) Untreated or chloroquine-treated (+CQ) HaCat cells stably expressing MP1 or MP1-LC3 were stained with anti-MP1 antiserum and DAPI for DNA content and were analyzed by confocal microscopy. Scale bars: 10 μm. Representative fields from one experiment are shown. (d) MDAMC cells stably expressing GFP-LC3 were infected with lentivirus encoding for MP1 or MP1-LC3. To inhibit degradation of GFP-LC3 and MP1 proteins in lysosomes, cells were treated with 50 μM CQ for 10 h and then stained with anti-MP1 antiserum and analyzed by confocal microscopy. Scale bars: 10 μm. Representative fields from one experiment are shown.

FIGS. 7 A-C demonstrate the characterization of Influenza MP 1 specific CD4+ and CD8+ T cell clones. (a) CD4 and CD8 expression of the clones was analyzed by flow cytometry. Clones 9.26, 11.46 and 10.9 were homogenously CD4+CD8− and clone 9.2 homogenously CD8+CD4−. (b) Their recognition of Influenza MP1 peptides was tested by IFNγ ELISPOT assays. The MP1 peptide library was divided in 6 subpools covering MP1 amino acid positions 1-51 (pool I), 41-88 (pool II), 78-128 (pool III), 118-163 (pool IV), 152-203 (pool V) and 193-252 (pool VI). Clones 92, 9.26 and 10.9 responded specifically to pool II and clone 11.46 to pool M. In addition, the CD8+ T cell clone 9.2, but not the CD4+ T cell clones, recognized the HLAA2 restricted MP1 epitope 58-66. Error bars indicate standard deviations. (c) MP1-specific CD4+ T cell clones were tested for recognition of individual peptides covering MP1 amino acid sequence 29-128, including all peptides of MP1 pools II and III. Clones 9.26 and 10.9 specifically recognized peptide epitope MP162-72 and clone 11.46 was specific for epitope MP1103-113. Error bars indicate standard deviations.

FIGS. 8 A-C demonstrate the fusion of MP1 to LC3 enhances CD4+ T cell recognition, while leaving CD8+ T cell recognition unaffected. (a) IFNγ-treated target cells (HaCat pulsed with cognate peptide, HaCat, or HaCat expressing GFP-LC3, MP1 or MP1-LC3) or untreated control cells were cocultured with the MP1-specific CD4+ T cell clones 9.26 (upper panel), 10.9 (middle panel) at effector to target (E:T) ratios of 2, 5 and 12.5, or clone 11.46 (lower panel) at effector to target (E:T) ratios of 10, 20 and 40. To exclude exogenous processing of MP1, IFNγ-treated HaCat cells were incubated with T cell clones in the presence of HLA-mismatched MP1 or MP1-LC3 expressing MDAMC cells. After 24 h of coculture, IFNγ in 1:2 diluted culture supernatants was measured by ELISA. Error bars indicate standard deviations and P-values for paired student's T test statistics across all E:T ratios are shown. One of two experiments each is shown. (b) Surface MHC class II staining on target cells used in the CD4+ T cell assay in (a). One of two experiments is shown. (c) IFNγ-treated or untreated target cells (MDAMC pulsed with cognate peptide, MDAMC or MDAMC expressing GFP-LC3, MP1 or MP1-LC3) were cocultured with the MP1-specific CD8+ T cell clone 9.2 (upper panel) at effector to target (E:T) ratios of 2, 5 and 12.5; clone 10.9 (middle panel) at effector to target (E:T) ratios of 2, 5 and 10; and clone 11.46 (lower panel) at effector to target (E:T) ratios of 5, 10 and 20. IFNγ in 1:20 diluted culture supernatants was measured by ELISA. Error bars indicate standard deviations. One of two experiments each is shown.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

While autophagic pathways have been suggested to be involved in endogenous MHC class II antigen processing, the degree to which autophagy is constitutively active in MHC class II positive antigen presenting cells (APCs) was not heretofore known, nor was it clear how efficient these pathways are for antigen delivery to the MHC class II loading compartment, and that such a pathway may be exploited for specific antigen delivery to the autophagosomal compartment, for presentation on MHC class II.

Autophagosomes were found, as exemplified herein, to constitutively fuse with MHC class II loading compartments in epithelial cells and targeting of this pathway via fusion of a protein or peptide of interest to the autophagosomal marker LC3, resulted in a strong increase in MHC class II presentation and CD4+ T cell recognition of the peptide, for example, as exemplified herein with influenza MP1 fusion constructs.

Such fusion proteins can be prepared via introduction of a nucleic acid, or vector comprising the same, encoding for the fusion protein.

In one embodiment, a nucleic acid is provided encoding a peptide or protein of interest fused in frame to a nucleic acid encoding an autophagosomal targeting protein, or a functional fragment thereof, wherein said peptide or protein of interest is poorly or not presented efficiently on a major histocompatibility complex (MHC) class II molecule.

### Nucleic Acids:

In one embodiment, the term “nucleic acid” molecule can include, but is not limited to, prokaryotic sequences, eukaryotic in RNA, cDNA from eukaryotic mRNA, genomic DNA sequences from eukaryotic (e.g., mammalian) DNA, and even synthetic DNA sequences. The term also refers to sequences that include any of the known base analogs of DNA and RNA.

As will be appreciated by one skilled in the art, a fragment or derivative of a nucleic acid sequence or gene that encodes for a protein or peptide can still function in the same manner as the entire, wild type gene or sequence. Likewise, forms of nucleic acid sequences can have variations as compared to wild type sequences, nevertheless encoding a protein or peptide, or fragments thereof, retaining wild type function exhibiting the same biological effect, despite these variations. Each of these represents an embodiment herein.

The nucleic acids embodied herein can be produced by any synthetic or recombinant process, such as is well known in the art. Nucleic acids according to the teachings herein can further be modified to alter biophysical or biological properties by means of techniques known in the art. For example, the nucleic acid can be modified to increase its stability against nucleases (e.g., “end-capping”), or to modify its lipophilicity, solubility, or binding affinity to complementary sequences.

DNA according to the teachings herein can also be chemically synthesized by methods known in the art. For example, the DNA can be synthesized chemically from the four nucleotides in whole or in part by methods known in the art. Such methods include those described in Caruthers (1985). DNA can also be synthesized by preparing overlapping double-stranded oligonucleotides, filling in the gaps, and ligating the ends together (see, generally, Sambrook et al. (1989) and Glover et al. (1995)). DNA expressing functional homologs of the protein can be prepared from wild-type DNA by site-directed mutagenesis (see, for example, Zoller et al. (1982); Zoller (1983); and Zoller (1984); McPherson (1991)). The DNA obtained can be amplified by methods known in the art. One suitable method is the polymerase chain reaction (PCR) method described in Saiki et al. (1988), Mullis et al., U.S. Pat. No. 4,683,195, and Sambrook et al. (1989).

The nucleic acid molecules embodied herein comprise a peptide or protein of interest fused in frame to a nucleic acid encoding an autophagosomal targeting protein, or a functional fragment thereof.

In one embodiment, the autophagosomal targeting protein is an LC3 protein.

In one embodiment, the LC3 protein is encoded by nucleic acid having a sequence corresponding to, or homologous to, that disclosed in NCBI's Entrez nucleotide database, having the Accession number: NM_{—}025735, NM_{—}142392, NM_{—}167245, BC018634, AY619720, 13C086389, BC045759, BC067797, BC010596, BC018634, BC083556, AF303888, AF183417, or a homologue thereof.

In another embodiment, the LC3 protein has an amino acid sequence corresponding to, or homologous to, that disclosed in NCBI's Entrez protein database, having the Accession number: Q9GZQ8, Q62625, AAU04437, NP_{—}852610, NP_{—}073729, NP 955794, AAM10499, NP_{—}080011, AAH18634, AAH86389, AAH83556, AAH58144, AAP36120, AA039078, or a homologue thereof.

The mammalian microtubule-associated protein light chain 3 (LC3) and homologues thereof, such as yeast Atg8, are essential components of autophagy. In rats, following synthesis, the C-terminus of LC3 has been shown to be cleaved by a cysteine protease-Atg4, to produce LC3-I, which is located in a cytosolic fraction. LC3-I can be converted to LC3-II through the processing by Atg7 (E1-like enzyme) and Atg3 (E2-like enzyme). LC3-II is modified by phosphatidylethanolamine on its C-terminus and binds tightly to the autophagosomal membrane. Splice variants of rat LC3, have been found, as well, for example, LC3A and LC3B, respectively, and subcellular localization studies showed that both LC3A and LC3B are colocalized with LC3 and associated with the autophagic membranes. Such splice variants, or associated proteins may, in turn be used in the methods, nucleic acids, constructs, cells and compositions embodied herein, as autophagosomal targeting proteins, in order to target linked proteins to the Class II processing and presentation machinery, as described herein. It is to be understood that any protein identified, which is found associated with autophagosomes, and which, when prepared, as a fusion construct with a protein or peptide of interest, serves to target the construct to an autophagosome, and/or facilitate presentation of the protein or peptide of interest, or a fragment thereof, in the context of MHC class II, is embodied herein. Such protein may be a homologue of previously identified autophagosme-associated proteins.

The term “homologue” or “homology”, in some embodiments, indicates a percentage of amino acid or nucleotide residues in the candidate sequence that are identical with the residues of a corresponding native sequence, which, in one embodiment, may be after aligning the sequences and introducing gaps, if necessary, to achieve the maximum percent homology, and in some embodiments, not considering any conservative substitutions as part of the sequence identity. In some embodiments, neither N- or C-terminal extensions nor insertions are construed as reducing identity or homology. Methods and computer programs for the alignment are well known in the art.

Homology may be determined, in some embodiments, by computer algorithm for sequence alignment, by methods well described in the art. For example, computer algorithm analysis of nucleic acid sequence homology may include the utilization of any number of software packages available, such as, for example, the BLAST, DOMAIN, BEAUTY (BLAST Enhanced Alignment Utility), GENPEPT and TREMBL packages.

In other embodiments, determining homology is via determination of candidate sequence hybridization, methods of which are well described in the art (See, for example, “Nucleic Acid Hybridization” Hames, B. D., and Higgins S. J., Eds. (1985); Sambrook et al., 1989, Molecular Cloning, A Laboratory Manual, (Volumes 1-3) Cold Spring Harbor Press, N.Y.; and Ausubel et al., 1989, Current Protocols in Molecular Biology, Green Publishing Associates and Wiley Interscience, N.Y). For example methods of hybridization may be carried out under moderate to stringent conditions, to the complement of a DNA encoding an autophagosomal targeting protein. Hybridization conditions being, for example, overnight incubation at 42° C. in a solution comprising: 10-20% formamide, 5×SSC (150 mM NaCl, 15 mM trisodium citrate), 50 mM sodium phosphate (pH 7.6), 5×Denhardt's solution, 10%® dextran sulfate, and 20 μg/ml denatured, sheared salmon sperm DNA.

As used herein, the terms “homology”, “homologue” or “homologous”, in any instance, indicate that the sequence referred to, whether an amino acid sequence, or a nucleic acid sequence, exhibits, in one embodiment at least 70% correspondence with the indicated sequence. In another embodiment, the amino acid sequence or nucleic acid sequence exhibits at least 72% correspondence with the indicated sequence. In another embodiment, the amino acid sequence or nucleic acid sequence exhibits at least 75% correspondence with the indicated sequence. In another embodiment, the amino acid sequence or nucleic acid sequence exhibits at least 80% correspondence with the indicated sequence. In another embodiment, the amino acid sequence or nucleic acid sequence exhibits at least 82% correspondence with the indicated sequence. In another embodiment, the amino acid sequence or nucleic acid sequence exhibits at least 85% correspondence with the indicated sequence. In another embodiment, the amino acid sequence or nucleic acid sequence exhibits at least 87% correspondence with the indicated sequence. In another embodiment, the amino acid sequence or nucleic acid sequence exhibits at least 90% correspondence with the indicated sequence. In another embodiment, the amino acid sequence or nucleic acid sequence exhibits at least 92% correspondence with the indicated sequence. In another embodiment, the amino acid sequence or nucleic acid sequence exhibits at least 95% or more correspondence with the indicated sequence. In another embodiment, the amino acid sequence or nucleic acid sequence exhibits at least 97% correspondence with the indicated sequence. In another embodiment, the amino acid sequence or nucleic acid sequence exhibits at least 99% correspondence with the indicated sequence. In another embodiment, the amino acid sequence or nucleic acid sequence exhibits 95%-100% correspondence with the indicated sequence. Similarly, as used herein, the reference to a correspondence to a particular sequence includes both direct correspondence, as well as homology to that sequence as herein defined.

Homology, as used herein, may refer to sequence identity, or may refer to structural identity, or functional identity. By using the term “homology” and other like forms, it is to be understood that any molecule, whether nucleic acid or peptide, that functions similarly, and/or contains sequence identity, and/or is conserved structurally so that it approximates the reference sequence, is embodied herein.

Thus, in one embodiment, the nucleic acid encoding an autophagosomal targeting protein has a sequence homologous to, or corresponding to SEQ ID NO: 1.

In another embodiment, the autophagosomal targeting protein is a gamma-aminobutyric-acid-type-A-receptor-associated protein (GABARAP), Golgi-associated ATPase enhancer of 16 kDa (GATE16), or a homologue thereof.

In one embodiment, the gamma-aminobutyric-acid-type-A-receptor-associated protein (GABARAP) protein is encoded by nucleic acid having a sequence corresponding to, or homologous to, that disclosed in NCBI's Entrez nucleotide database, having the Accession number: NM_{—}174874, NM_{—}007278, BC106748, NM_{—}172036, NM_{—}019749, BC058441, BC002126, AF161588, AF161587, NM_{—}177408, or a homologue thereof.

In another embodiment, the gamma-aminobutyric-acid-type-A-receptor-associated protein (GABARAP) protein has an amino acid sequence corresponding to, or homologous to, that disclosed in NCBI's Entrez protein database, having the Accession number: CAG47031, CAG33324, NP_{—}062723, AAD47643, Q9GJW7, AAI06749, CAI35162, AAH30350, or a homologue thereof.

In one embodiment, the Golgi-associated ATPase enhancer of 16 kDa (GATE16) protein is encoded by nucleic acid having a sequence corresponding to, or homologous to, that disclosed in NCBI's Entrez nucleotide database, having the Accession number: AY117147, NM 026693, NM_{—}031412, BC081436, or a homologue thereof.

In another embodiment, the Golgi-associated ATPase enhancer of 16 kDa (GATE16) protein has an amino acid sequence corresponding to, or homologous to, that disclosed in NCBI's Entrez protein database, having the Accession number: AAM77036, P60519, BAB21549, to BAB21548, P60520, NP_{—}080969, NP 495277, or a homologue thereof.

In another embodiment, the autophagosomal targeting protein is the KFERQ signal sequence from RNAse A for chaperone mediated autophagy, and has the nucleic acid sequence 5′ aaattcgagcggcag3′ corresponding to, or homologous to, that disclosed in NCBI's Entrez nucleotide database, having the Accession number NM_D26129, or a homologue thereof.

In another embodiment, the KFERQ signal sequence has the amino acid sequence KFERQ corresponding to, or homologous to, that disclosed in NCBI's Entrez protein database, having the Accession number: NP_D26129, or a homologue thereof.

In another embodiment, the autophagosomal targeting protein is the GA repeat domain from the Epstein Barr virus nuclear antigen 1 (EBNA 1) sequence (aa268-984), and has a nucleic acid sequence corresponding to, or homologous to, that disclosed in EMBL nucleotide database, having the Accession number EMBL-EBI_CAA24816, or a homologue thereof.

In another embodiment, the GA repeat domain of EBNA1 has an amino acid sequence corresponding to, or homologous to aa268-984 of the EBNA1 amino acid sequence, that is disclosed in EMBL protein database, having the Accession number: EMBL-EBI_CAA24816, or a homologue thereof.

The nucleic acids embodied herein comprise sequences encoding autophagosomal targeting proteins, fused in frame to those encoding a protein or peptide of interest, wherein the protein or peptide of interest is underpresented, poorly presented, or not presented at all, in the context of a major histocompatibility complex (MHC) class II protein.

### Antigens:

In one embodiment, the protein or peptide of interest, or fragment thereof, comprise an epitope whose presentation specifically on MHC class II is desired. In one embodiment, the term “epitope” refers to an immunogenic amino acid sequence. An epitope may refer to a minimum amino acid sequence of 6-8 amino acids (i.e., a peptide), which minimum sequence is immunogenic, when removed from its natural context. An epitope also may refer, in other embodiments, to that portion of a natural polypeptide which is immunogenic, where the natural polypeptide containing the epitope is referred to as an antigen. In some embodiments, a polypeptide or antigen may contain one or more distinct epitopes. An epitope may refer, in some embodiments, to an immunogenic portion of a multichain polypeptide, i.e., which is encoded by distinct open reading frames. The terms epitope, peptide, and polypeptide all refer to a series of amino acids connected one to the other by peptide bonds between the alpha-amino and alpha-carboxy groups of adjacent amino acids, and may contain or be free of modifications such as to glycosylation, side chain oxidation, or phosphorylation, provided such modifications, or lack thereof, do not destroy immunogenicity. As used herein, the term “peptide” is meant to refer to both a peptide and a polypeptide or protein.

In some embodiments, the epitope (peptide, polypeptide, antigen) is as small as possible while still maintaining immunogenicity. Immunogenicity is indicated by the ability to elicit an immune response, as described herein, for example, by the ability to bind an MHC class II molecule and to induce a T cell response, e.g., by measuring T cell cytokine production.

In some embodiments, the terms “antigen” or “immunogen” refer to a peptide, protein, polypeptide which is immunogenic, that is capable of eliciting an immune response in a mammal, and therefore contains at least one and may contain multiple epitopes. As embodied herein, a “pathogen”, organism, or “agent” may cause a disease or disorder, for which the methods, cells, nucleic acids, vectors and/or compositions embodied herein are used. In some embodiments, reference to pathogen or organism, refers to a virus, bacteria, fungus, or a parasite. The term “agent” also may refer to antigens such as tumor antigens or antigens associated with auto-immunity or transplant, for example, self (host) antigens or graft antigens.

In one embodiment, the peptide or protein of interest is virally encoded, for example, by a vaccinia virus or lentivirus. In another embodiment, the peptide or protein of interest is encoded by the influenza virus, which in another embodiment, is a matrix protein, and in another embodiment, the nucleic acid encoding an autophagosomal targeting protein fused in frame to an influenza matrix protein, has a sequence homologous to, or corresponding to SEQ ID NO: 2.

In one embodiment, the peptide or protein of interest is derived from a virus, which is a member of the following viral families: Retroviridae (e.g., human immunodeficiency viruses, such as HIV-1 (also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g. influenza viruses); Bungaviridae (e.g., Hantaan viruses, bunga viruses, phleboviruses and Nairo viruses); Arenaviridae (hemorrhagic fever viruses); Reoviridae (erg., reoviruses, orbiviurses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herpesviridae (herpes simplex virus (HSV) 1 and 2, varicella zoster virus, cytomegalovirus (CMV), herpes viruses'); Poxyiridae (variola viruses, vaccinia viruses, pox viruses); Hepatitis X, Epstein-Barr Virus, herpes simplex viruses, and Iridoviridae (e.g. African swine fever virus); and unclassified viruses (e.g., the etiological agents of Spongiform encephalopathies, the agent of delta hepatities (thought to be a defective satellite of hepatitis B virus), the agents of non-A, non-B hepatitis (class 1=internally transmitted; class 2=parenterally transmitted (i.e., Hepatitis C); Norwalk and related viruses, and astroviruses).

In one embodiment, the peptide or protein of interest is derived from a bacterium, which is an intracellular bacteria, which may include, inter alia: Shigella sp., Salmonella sp., Francisella sp., Helicobacter sp., including Helicobacter pylori, Borellia burgdorferi, Legionella sp. including Legionella pneumophilia, Mycobacterium sp. (e.g. M. tuberculosis, M. avium, M. intracellulare, M. kansaii, M. gordonae), Staphylococcus sp., including Staphylococcus aureus, Neisseria sp., including Neisseria gonorrhoeae, Neisseria meningitidis, Listeria sp., including Listeria monocytogenes, Streptococcus sp., including, inter-alia: Streptococcus pyogenes (Group A Streptococcus), Streptococcus agalactiae (Group B Streptococcus), Streptococcus viridans group, Streptococcus faecalis, Streptococcus bovis, Streptococcus anaerobic sp., Streptococcus pneumoniae, pathogenic Campylobacter sp., Enterococcus sp., Chlamydia sp., Haemophilus influenzae, Bacillus anthracis, Corynebacterium sp., Corynebacterium diphtheriae, Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema pallidium, Treponema pertenue, Leptospira, Actinomyces israelli, Francisella tularensis, or others known in the art (See G. L. Mandell, “Introduction to Bacterial Disease” in Cecil Textbook Of Medicine, (W.B. Saunders Co., 1996) 1556-7).

In one embodiment, the peptide or protein of interest is derived from a Protozoa, which may include, inter alia: Plasmodium (e.g., Plasmodium falciparum, P. vivax, P. ovale and P. malariae), Trypanosoma, Toxoplasma, Leishmania, Cryptosporidium, and others known in the art.

In one embodiment, the peptide or protein of interest is derived from a fungus, which may include, inter alia: Cryptococcus neoformans, Histoplasma capsulatum, Coccidioides immitis, Blastomyces dernzatitidis, Chlamydia trachomatis, Candida albicans.

### Cancer Antigens:

In one embodiment, the peptide or protein of interest is derived from a neoplastic or cancerous cell or tissue, or preneoplastic cell or tissue. In one embodiment, the cancerous cell may be a malignant, or, in another embodiment, a non-malignant cancer. Cancers or tumors may include, but are not limited to biliary tract cancer; brain cancer; breast cancer; cervical cancer; choriocarcinoma; colon cancer; endometrial cancer; esophageal cancer; gastric cancer; intraepithelial neoplasms; lymphomas; liver cancer; lung cancer (e.g. small cell and non-small cell); melanoma; neuroblastomas; oral cancer; ovarian cancer; pancreas cancer; prostate cancer; rectal cancer; sarcomas; skin cancer; testicular cancer; thyroid cancer; and renal cancer, as well as other carcinomas and sarcomas. In one embodiment the cancer is hairy cell leukemia, chronic myelogenous leukemia, cutaneous T-cell leukemia, multiple myeloma, follicular lymphoma, malignant melanoma, squamous cell carcinoma, renal cell carcinoma, prostate carcinoma; bladder cell carcinoma, or colon carcinoma.

In another embodiment, the antigens are derived from canerous cells occurring in the adrenal glands; bladder; bone; breast; cervix; endocrine glands (including thyroid glands, the pituitary gland, and the pancreas); colon; rectum; heart; hematopoietic tissue; kidney; liver; lung; muscle; nervous system; brain; eye; oral cavity; pharynx; larynx; ovaries; penis; prostate; skin (including melanoma); testicles; thymus; and uterus. Examples of such tumors include apudoma, choristoma, branchioma, malignant carcinoid syndrome, carcinoid heart disease, carcinoma (e.g., Walker, basal cell, basosquamous, Brown-Pearce, ductal, Ehrlich tumor, in situ, Krebs 2, Merkel cell, mucinous, non-small cell lung, oat cell, papillary, scirrhous, bronchiolar, bronchogenic, squamous cell, and transitional cell), plasmacytoma, melanoma, chondroblastoma, chondroma, chondrosarcoma, fibroma, fibrosarcoma, giant cell tumors, histiocytoma, lipoma, liposarcoma, mesothelioma, myxoma, myxosarcoma, osteoma, osteosarcoma, Ewing's sarcoma, synovioma, adenofibroma, adenolymphoma, carcinosarcoma, chordoma, mesenchymoma, mesonephroma, myosarcoma, ameloblastoma, cementoma, odontoma, teratoma, thymoma, trophoblastic tumor, adenocarcinoma, adenoma, cholangioma, cholesteatoma, cylindroma, cystadenocarcinoma, cystadenoma, granulosa cell tumor, gynandroblastoma, hepatoma, hidradenoma, islet cell tumor, Leydig cell tumor, papilloma, Sertoli cell tumor, theca cell tumor, leiomyoma, leiomyosarcoma, myoblastoma, myoma, myosarcoma, rhabdomyoma, rhabdomyosarcoma, ependymoma, ganglioneuroma, glioma, medulloblastoma, meningioma, neurilemmoma, neuroblastoma, neuroepithelioma, neurofibroma, neuroma, paraganglioma, paraganglioma nonchromaffin, angiokeratoma, angiolymphoid hyperplasia with eosinophilia, angioma sclerosing, angiomatosis, glomangioma, hemangioendothelioma, hemangioma, hemangiopericytoma, hemangiosarcoma, lymphangioma, lymphangiomyoma, lymphangiosarcoma, pinealoma, carcinosarcoma, chondrosarcoma, cystosarcoma phyllodes, fibrosarcoma, hemangiosarcoma, leiomyosarcoma, leukosarcoma, liposarcoma, lymphangiosarcoma, myosarcoma, myxosarcoma, ovarian carcinoma, rhabdomyosarcoma, sarcoma (e.g., Ewing's experimental, Kaposi's, and mast-cell), neoplasms and for other such cells.

In one embodiment, the cancer-associated antigen may be referred to as a tumor antigen, which in one embodiment, is a peptide or protein, associated with a tumor or cancer cell surface. Cancer antigens may represent an immunogenic portion of a tumor or cancer.

Cancer antigens comprise, in some embodiments, antigens that are normally silent (i.e., not expressed) in normal cells, or in other embodiments, those that are expressed only at certain stages of differentiation, or in other embodiments, those that are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as, for example, those carried on RNA and DNA tumor viruses. Examples of tumor antigens include MAGE, MART-1/Melan-A, gp100, Dipeptidyl peptidase IV (DPPIV), adenosine deaminase-binding protein (ADAbp), cyclophilin b, Colorectal associated antigen (CRC)-C017-1A/GA733, Carcinoembryonic Antigen (CEA) and its immunogenic epitopes CAP-1 and CAP-2, etv6, aml 1, Prostate Specific Antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MACE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LACE-I, NAG, GnT-V, MUM-I, CDK4, tyrosinase, p53, MUC family, HER2/neu, p21ras, RCAS1, α-fetoprotein, E-cadherin, α-catenin, (β-catenin and γ-catenin, p120ctn, gp100 Pme1117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, HA, EBV-encoded nuclear antigen (EBNA)-1, brain glycogen phosphorylase, SSX-1, SSX-2 (HOM-MEL-40), SSX-1, SSX-4, SSX-5, SCP-1 and CT-7, and c-erbB-2.

It is to be understood that any cell cancer antigen as herein described may be used in the methods and/or compositions embodied herein, and represents an embodiment thereof.

Cancer antigens for use in the nucleic acids, methods and compositions embodied herein may include, inter-alis, acute lymphoblastic leukemia (etv6; aml1; cyclophilin b), B cell lymphoma (Ig-idiotype), glioma (E-cadherin; α-catenin; β-catenin; γ-catenin; p120ctn), bladder cancer (p21ras), biliary cancer (p21ras), breast cancer (MUC family; HER2/neu; c-erbB-2), cervical carcinoma (p53; p21ras), colon carcinoma (p21ras; BER2/neu; c-erbB-2; MUC family), colorectal cancer (Colorectal associated antigen (CRC)-C017-1A/GA733; APC), choriocarcinoma (CEA), epithelial cell-cancer (cyclophilin b), gastric cancer (HER2/neu; c-erbB-2; ga733 glycoprotein), hepatocellular cancer (α-fetoprotein), Hodgkins lymphoma (Imp-1; EBNA-1), lung cancer (CEA; MAGE-3; NY-ESO-1), lymphoid cell-derived leukemia (cyclophilin b), melanoma (p15 protein, gp75, oncofetal antigen, GM2 and GD2 gangliosides), myeloma (MUC family; p21ras), non-small cell lung carcinoma (HER2/neu; c-erbB-2), nasopharyngeal cancer (Imp-1; EBNA-1), ovarian cancer (MUC family; HER2/neu; c-erbB-2), prostate cancer (Prostate Specific Antigen (PSA) and its immunogenic epitopes PSA-1, PSA-2, and PSA-3; PSMA; HER2/neu; c-erbB-2), pancreatic cancer (p21ras; MUC family; HER2/neu; c-erbB-2; ga733 glycoprotein), renal (HER2/neu; c-erbB-2), squamous cell cancers of cervix and esophagus (viral products such as human papilloma virus proteins), testicular cancer (NY-ESO-1), T cell leukemia (HTLV-1 epitopes), and melanoma (Melan-A/MART-1; cdc27; MAGE-3; p21 ras; gp100 Pme1117). Hyperplastic, preneoplastic or neoplastic cells expressing these tumor antigens may be used in the methods and/or compositions embodied herein. It is to be understood that any of the cancers described hereinabove may be accordingly treated with the nucleic acids, vectors, compositions and methods embodied herein, and represent an embodiment thereof.

The antigen encoded by nucleic acids and vectors embodied herein are endogenously synthesized and epitopes of the antigen fused to an autophagosomal targeting protein, targeted to an MHC class II loading compartment, whereupon ultimately, the epitope is displayed in association with Class II MHC molecules.

MHC class II molecules typically bind peptides 12-20 amino acids in length. The peptide flanking residues (PFRs) of these ligands extend from a central binding core consisting of nine amino acids. PFRs can alter the immunogenicity of T cell epitopes. Certain motifs have been associated with enhanced MHC class II binding, for example, the presence of C-terminal basic residues and N-terminal prolines in MHC class II ligands. Such motifs are considered, in some embodiments herein, in the design and preparation of the constructs and nucleic acids embodied herein, for tailoring the types of peptides and/or proteins which are targeted to the autophagosome, in accordance with the methods, molecules and compositions embodied herein.

In some embodiments, antigenic peptides are created by encoding a C-terminal region of the Ii-Key segment of the Ii protein fused in frame to the N-terminus of the peptide for MHC class II presentation, which in turn, may enhance potency of presentation of the MHC class II epitope.

In some embodiments, computer epitope prediction programs are used in the design of the nucleic acids embodied herein, for the construction of constructs and/or nucleic acids to encoding an epitope which will bind well to the MHC class II molecule, for better presentation on the molecule, once targeted to the autophagosome. Such prediction algorithms are known in the art, and may comprise, for example, those found on the following websites:

(http://syfpeithi.bmiheidelberg.com/scripts/MHCServer.dll/home.html)
(http://www.imtech.res.in/raghava/propred/index.html).

In another embodiment, the antigen may be any molecule recognized by the immune system of the subject, as foreign. The antigen may, in another embodiment, derives from a mammalian cell, an infectious virus, bacteria, fungi, or other organism (e.g., protists). These infectious organisms may be active, in one embodiment or inactive, in another embodiment, which may be accomplished, for example, through exposure to heat or removal of at least one protein or gene required for replication of the organism. In one embodiment, a nucleic acid encoding the antigenic protein or peptide is isolated, or in another embodiment, synthesized.

In another embodiment, a library of nucleic acids, encoding peptides that span an antigenic protein are used herein. In one embodiment, the nucleic acids encode peptides, which are about 15 amino acids in length, and may, in another embodiment, be constructed to encode a peptide staggered every 4 amino acids along the length of the antigenic protein. In another embodiment, the antigens are obtained by recombining two or more forms of a nucleic acid that encode a polypeptide of the antigen, for example, as derived from a pathogenic agent, or antigen involved in another disease or condition. These recombination methods, referred to in one embodiment, as “DNA shuffling”, use as substrates forms of the nucleic acid that differ from each other in two or more nucleotides, so a library of recombinant nucleic acids results. The library is then screened to identify at least one optimized recombinant nucleic acid that encodes an optimized recombinant antigen that has improved ability to induce an immune response to the pathogenic agent or other condition. The resulting recombinant antigens often are chimeric in that they are recognized by antibodies (Abs) reacting against multiple pathogen strains, and generally can also elicit broad-spectrum immune responses.

In other embodiments, the different forms of the nucleic acids that encode antigenic polypeptides are obtained from members of a family of related agents, for example, pathogenic agents. This scheme of performing DNA shuffling using nucleic acids from related organisms, known as “family shuffling,” is described in Crameri et al. ((1998) Nature 391: 288-291). Polypeptides of different strains and serotypes of pathogens generally vary between 60-98%, which will allow for efficient family DNA shuffling. Therefore, family DNA shuffling provides an effective approach to generate multivalent, crossprotective antigens. The recombinant fusion to proteins, as described and claimed herein, are then produced, by methods well known to those skilled in the art, and then used in the compositions and methods embodied herein.

### Vectors:

In one embodiment, a vector is provided comprising a nucleic acid embodied herein.

The nucleic acid sequences described herein may be subcloned within a particular vector, the choice of which may depend, in some embodiments, on the desired method of introduction, expression, regulation, etc. of the sequence within cells. To generate the nucleic acid constructs in context of the embodiments herein, the polynucleotide segments encoding sequences of interest can be ligated into commercially available expression vector systems suitable for transducing/transforming mammalian cells and for directing the expression of recombinant products within the transduced/transformed cells. It will be appreciated that such commercially available vector systems can easily be modified via commonly used recombinant techniques in order to replace, duplicate or mutate existing promoter or enhancer sequences and/or introduce any additional polynucleotide sequences such as for example, sequences encoding additional selection markers or sequences encoding reporter polypeptides.

In one embodiment, the term “vector” refers to a nucleic acid construct containing a sequence of interest that has been subcloned within the vector, in this case, the nucleic acid sequence encoding the fusion products as herein described.

A vector as embodied herein may include an appropriate selectable marker. The vector may further include an origin of replication, and may be a shuttle vector, which can propagate both in bacteria, such as, for example, E. coli (wherein the vector comprises an appropriate selectable marker and origin of replication) and be compatible for propagation in vertebrate cells, or integration in the genome of an organism of choice. The vector according to this the embodiments herein can be, for example, a plasmid, a bacmid, a phagemid, a cosmid, a phage, a virus or an artificial chromosome.

In some embodiments, the vectors embodied herein will have a regulatable promoter. The nucleotide sequences which regulate expression of a gene product (which are referred to herein as “regulatory elements”, for example, promoter and enhancer sequences) may be selected, in one embodiment, based upon the type of cell in which the gene product is to be expressed, or in another embodiment, upon the desired level of expression of the gene product.

For example, a promoter known to confer cell-type specific expression of a gene linked to the promoter can be used. A promoter specific for myoblast gene expression can be linked to a gene of interest to confer muscle-specific expression of that gene product. Muscle-specific regulatory elements which are known in the art include upstream regions from the dystrophin gene (Klamut et al., (1989) Mol. Cell. Biol. 9:2396), the creatine kinase gene (Buskin and Hauschka, (1989) Mol. Cell. Biol. 9:2627) and the troponin gene (Mar and Ordahl, (1988) Proc. Natl. Acad. Sci. USA. 85:6404).

Regulatory elements specific for other cell types are known in the art (e.g., the albumin enhancer for liver-specific expression; insulin regulatory elements for pancreatic islet cell-specific expression; various neural cell-specific regulatory elements, including neural dystrophin, neural enolase and A4 amyloid promoters). In another embodiment, a regulatory element, which can direct constitutive expression of a gene in a variety of different cell types, such as a viral regulatory element, can be used. Examples of viral promoters commonly used to drive gene expression include those derived from polyoma virus, Adenovirus 2, cytomegalovirus and Simian Virus 40, and retroviral LTRs.

In another embodiment, a regulatory element, which provides inducible expression of a gene linked thereto can be used. The use of an inducible regulatory element (e.g., an inducible promoeter) allows for modulation of the production of the gene product in the cell. Examples of potentially useful inducible regulatory systems for use in eukaryotic cells include hormone-regulated elements (e.g., see Mader, S, and White, J. H. (1993) Proc. Natl. Acad. Sci. USA 90:5603-5607), synthetic ligand-regulated elements (see, e.g., Spencer, D. M. et al 1993) Science 262:1019-1024) and ionizing radiation-regulated elements (e.g., see Manome, Y. Et al. (1993) Biochemistry 32:10607-10613; Datta, R. et al. (1992) Proc. Natl. Acad. Sci. USA89:1014-10153). Additional tissue-specific or inducible regulatory systems, may be developed for use in accordance with the embodiments herein.

In some embodiments, the vectors and nucleic acids embodied herein are introduced into cells, which in other embodiments comprise cells embodied herein. There are a number of techniques known in the art for introducing the above described recombinant vectors into cells as embodied herein, such as, but not limited to: direct DNA uptake techniques, and virus, plasmid, linear DNA or liposome mediated transduction, receptor-mediated uptake and magnetoporation methods employing calcium-phosphate mediated and DEAE-dextran mediated methods of introduction, electroporation, liposome-mediated transfection, direct injection, and receptor-mediated uptake (for further detail see, for example, “Methods in Enzymology” Vol. 1-317, Academic Press, Current Protocols in Molecular Biology, Ausubel F. M. et al. (eds.) Greene Publishing Associates, (1989) and in Molecular Cloning: A Laboratory Manual, 2nd Edition, Sambrook et al. Cold Spring Harbor Laboratory Press, (1989), or other standard laboratory. manuals). Bombardment with nucleic acid coated particles is also envisaged.

The efficacy of a particular expression vector system and method of introducing nucleic acid into a cell can be assessed by standard approaches routinely used in the art. For example, DNA introduced into a cell can be detected by a filter hybridization technique (e.g., Southern blotting) and RNA produced by transcription of introduced DNA can be detected, for example, by Northern blotting, RNase protection or reverse transcriptase-polymerase chain reaction (RT-PCR). The gene product can be detected by an appropriate assay, for example by immunological detection of a produced protein, such as with a specific antibody, or by a functional assay to detect a functional activity of the gene product, such as an enzymatic assay. If the gene product of interest to be expressed by a cell is not readily assayable, an expression system can first be optimized using a reporter gene linked to the regulatory elements and vector to be used. The reporter gene encodes a gene product, which is easily detectable and, thus, can be used to evaluate efficacy of the system. Standard reporter genes used in the art include genes encoding β-galactosidase, chloramphenicol acetyl transferase, luciferase and human growth hormone, or any of the marker proteins listed herein.

In some embodiments, the vector is a viral vector such as but not limited to a vaccinia virus or lentivirus. In other embodiments, a packaging system is constructed, comprising cDNA encoding an autophagosomal targeting protein, and the protein or peptide of interest.

A packaging system is a vector, or a plurality of vectors, which collectively provide in expressible form all of the genetic information required to produce a virion which can encapsidate the nucleic acid, transport it from the virion-producing cell, transmit it to a target cell, and, in the target cell, facilitate transgene expression. In some embodiments, the packaging system is substantially incapable of packaging itself, hence providing a means of attenuation, since virion production, following introduction into target cells is prevented.

In another embodiment, the recombinant vectors contemplated herein further comprise an insertion of a heterologous nucleic acid sequence encoding a marker polypeptide. The marker polypeptide may comprise, for example, green fluorescent protein (GFP), DS-Red (red fluorescent protein), secreted alkaline phosphatase (SEAP), beta-galactosidase, luciferase, or any number of other reporter proteins known to one skilled in the art.

In another embodiment, the recombinant vectors and nucleic acids embodied herein may further encode for an immunomodulating protein.

Examples of useful immunomodulating proteins include cytokines or chemokines. Useful examples include GM-CSF, IL-2, IL-12, IL-4, IFN-γ, or a combination thereof. Further useful examples include interleukins for example interleukins 1 to 15, interferons alpha, beta or gamma, tumour necrosis factor, granulocyte-macrophage colony stimulating factor (GM-CSF), macrophage colony stimulating factor (M-CSF), granulocyte colony stimulating factor (G-CSF), chemokines such as neutrophil activating protein (NAP), macrophage chemoattractant and activating factor (MCAF), RANTES, macrophage inflammatory peptides MIP-1a and MIP-1b, or a combination thereof.

In another embodiment, the immunomodulatory proteins may be of human or non-human animal specificity, and may comprise extracellular domains and/or other fragments with comparable binding activity to the naturally occurring proteins. Immunomodulatory proteins may, in another embodiment, be variants or analogs of the proteins described, and may be expressed comprise fusion proteins, or independently, as will be appreciated by one skilled in the art. The immunomodulating protein may be expressed, in some embodiments, concurrently with expression of the nucleic acids or vectors embodied herein, or in another embodiment, prior to, or in another embodiment, following expression of the nucleic acids or vectors embodied herein. Multiple immunomodulatory proteins may be incorporated within a single construct, and as such, represents an additional embodiment herein.

In another embodiment, a cell is provided comprising the nucleic acids embodied herein. In another embodiment, a cell is provided comprising the vectors embodied herein.

In one embodiment, a method is provided for stimulating or enhancing presentation of a peptide or protein of interest in the context of a major histocompatibility (MHC) class II molecule, the method comprising contacting a cell capable of expressing a major histocompatibility complex (MHC) class II molecule with a nucleic acid encoding a peptide or protein of interest fused in frame to a nucleic acid encoding an autophagosomal targeting protein, or a functional fragment thereof, whereby said autophagosomal targeting protein or a functional fragment thereof targets said peptide or protein of interest to an autophagosome, and said peptide, or a fragment of said protein of interest is displayed on the surface of said cell in the context of a major histocompatibility complex (MHC) class II molecule.

In one embodiment, the phrase “capable of expressing a major histocompatibility complex (MHC) class II molecule” refers to a cell endogenously expressing the molecule, or in another embodiment, a cell induced to express the molecule, or in another embodiment, engineered to express the molecule. In some embodiments, the cell is a so-called professional antigen presenting cell (APC), and may comprise a monocyte, a macrophage, or any cell of the myeloid lineage, a dendritic cell, a B cell, an M cell, or any combination thereof. In some embodiments, the cell is an epithelial cell, which, in some embodiments, is exposed to a cytokine, which in turn upregulates expression of the major histocompatibility complex (MHC) class II molecule, for example, following administration of interferon-γ.

### Cells and Targeted Therapy

In some embodiments, the cells are in a subject, who is healthy, or in another embodiment, are isolated from a subject who is healthy. In some embodiments, the methods/cells embodied herein are a means of vaccination, or in another embodiment, prevention, or in another embodiment, treatment of a disease.

In some embodiments, the cell is diseased and/or abnormal. The diseased or abnormal cells contemplated include, inter-alia: infected cells, neoplastic cells, pre-neoplastic cells, inflammatory foci, benign tumors or polyps, cafe au lait spots, leukoplakia, and other skin moles.

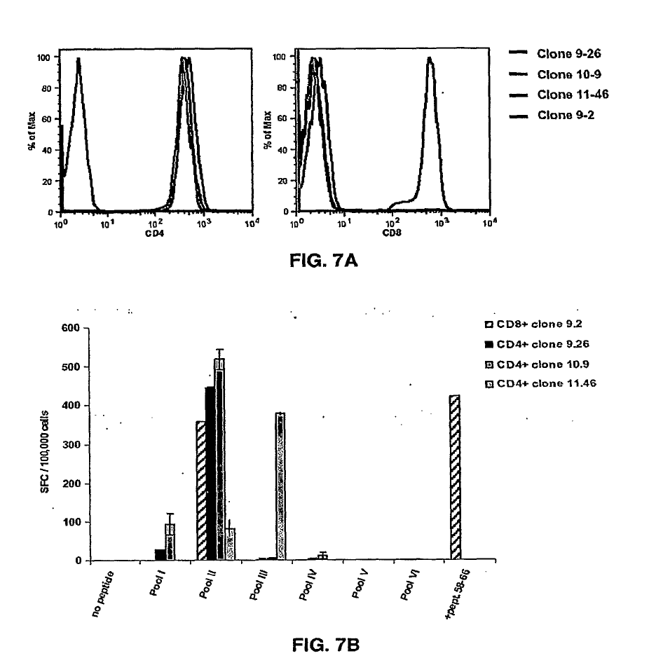

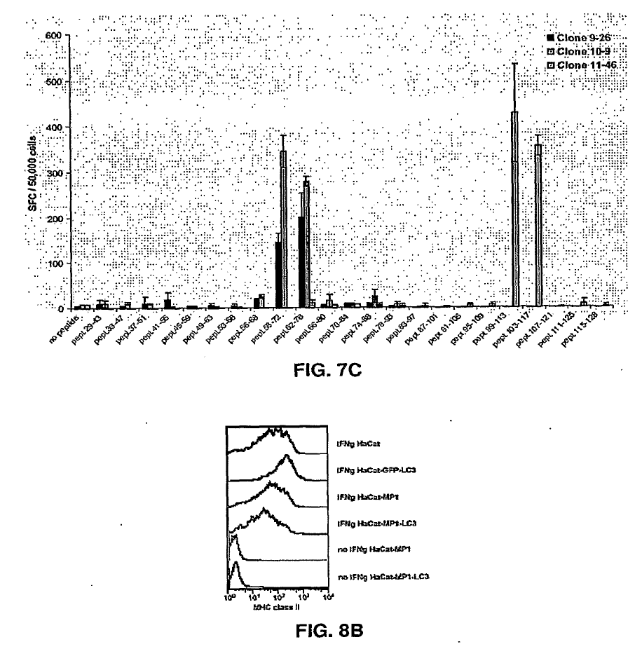

Influenza MP1 was targeted for enhanced MHC class II presentation, as exemplified herein, by its fusion to the autophagosome-associated Atg8/LC3 protein. Although access of MP1 to MHC class II presentation was shown when the antigen source was delivered by an intracellular route (FIG. 7), fusion to Atg8/LC3 significantly increased MHC class II presentation of MP1 by up to 17 fold. This increase occurred in spite of the fact that the expression of the MP1/LC3 fusion protein did not increase the total expression of MP1 or surface MHC class II.

Therefore, the targeting by autophagy was shown herein to be harnessed to boost MHC class II presentation and CD4⁺ T cell stimulation of antigens after intracellular delivery via for example viral vectors. The Atg8/LC3 fusion protein of MP1 used in this study was also efficiently processed onto MHC class I. Since it has been demonstrated that CD4⁺ T cells in addition to the above outlined direct anti-viral function are essential for the maintenance of protective CD8⁺ T cell effector functions and memory, improved stimulation of helper T cells in some embodiments, serves as a component of the immune response, whose provocation is desired. In some embodiment, such use is part of a broader vaccine development strategy, for example, via the development of recombinant viral vaccines.

In one embodiment, the cells embodied herein, or for use in any method embodied herein, are infected, and in one embodiment, the cells are infected with a virus, which, in one embodiment is influenza or, in another embodiment, HIV, or in another embodiment, any pathogen as described herein, or as will be known to one skilled in the art.

In one embodiment, the peptide or protein of interest is virally encoded, in one embodiment, by the influenza virus. In one embodiment, the peptide or protein of interest is a matrix protein, and in one embodiment, the nucleic acid according to this aspect, has a sequence homologous to, or corresponding to SEQ ID NO: 2.

In one embodiment, the cell is infected with a bacteria, which in one embodiment, is a mycobacteria, which in some embodiments, has been shown to provoke little MHC class II presentation, when unactivated. In some embodiments, the cell is infected with any bacteria, or pathogen, as described herein, or as will be known to one skilled in the art.

In another embodiment, the cell is neoplastic or preneoplastic.

In some embodiments, the cell is healthy, and promotes presentation of antigens as a preventive vaccine strategy. In another embodiment, the cells is healthy, and promotes presentation of antigens on MHC class II, as a prophylactic therapy, for a disease or condition, distal to the site of exposure of the healthy cell to the antigen.

In one embodiment, a method is provided for stimulating or enhancing an immune response in a subject, the method comprising contacting a cell capable of expressing a major histocompatibility complex (MHC) class II molecule in said subject with a nucleic acid encoding a peptide or protein of interest fused in frame to a nucleic acid encoding an autophagosomal targeting protein, or a functional fragment thereof, whereby said autophagosomal targeting protein or functional fragment thereof targets said peptide or protein of interest to an autophagosome, and said peptide, or a fragment of said protein of interest is displayed on the surface of said cell in the context of a major histocompatibility complex (MHC) class II molecule.

According to this aspect and in one embodiment, the cell is contacted indirectly with the nucleic acid or vector comprising the same. In one embodiment, the nucleic acid or vector comprising the same is administered intravenously to the subject, and in another embodiment, the subject is administered a composition comprising said nucleic acid or vector or cell comprising the same. In one embodiment, the composition is administered repeatedly, over a course of time. In one embodiment, the composition comprises a neoplastic cell isolated from the subject, which in one embodiment, is contacted ex vivo with the nucleic acid or vector comprising the same. In one embodiment, the subject has preneoplastic or hyperplastic cells or tissue, or in another embodiment, the subject is predisposed to neoplasia.

In some embodiments, the cells for use according to the embodiments herein, when administered to a subject, are autologous, or in another embodiment, syngeneic, or in another embodiment, allogeneic, with respect to the subject to which the cells are administered. In one embodiment, the cells are isolated from a subject having or predisposed to having neoplasia.

In one embodiment, the methods embodied herein may further employ the addition of cytokines or growth factors to the cells as described herein, or in another embodiment, may comprise the compositions embodied herein. In one embodiment, the cytokines and/or growth factors may to serve to enhance, activate, or direct the developing immune response stimulated in the subject, by the administration of the compositions or cells as herein described. In one embodiment, the cytokines and/or growth factors further promote maturation of the cells, which, in another embodiment, result in more robust presentation in the subject. In some embodiments, the cytokines bias the response, in terms of a Th-1 versus Th2, or vice versa, -type response.

In some embodiments, the cells are obtained from in vivo sources, such as, for example, most solid tissues in the body, peripheral blood, lymph nodes, gut associated lymphoid tissue, spleen, thymus, skin, sites of immunologic lesions, e.g. synovial fluid, pancreas, cerebrospinal fluid, tumor samples, granulomatous tissue, or any other source where such cells may be obtained. In one embodiment, the cells are obtained from human sources, which may be, in another embodiment, from human fetal, neonatal, child, or adult sources. In another embodiment, the cells used in the methods and/or compositions embodied herein may be obtained from animal sources, such as, for example, porcine or simian, or any other animal of interest. In another embodiment, cells used in the methods and/or compositions embodied herein may be obtained from subjects that are normal, or in another embodiment, diseased, or in another embodiment, susceptible to a disease of interest, or in another embodiment, of a particular genetic profile, such as, for example, from an individual which is known to overexpress a particular gene, or in another embodiment, underexpress a particular gene, or in another embodiment, be from a population typically susceptible to a given neoplasia.

In one embodiment, the term “contacting a cell” refers herein to both direct and indirect exposure of cell to the indicated item. In one embodiment, contact of cells with a nucleic acid or vector embodied herein, and optionally with a cytokine, growth factor, or combination thereof, is direct or, in another embodiment, indirect. In one embodiment, contacting a cell may comprise direct injection of the cell through any means well known in the art, such as microinjection. It is also envisaged, in another embodiment, that the supply to the cell is indirect, such as via provision in a culture medium that surrounds the cell, or administration to a subject, via any route well known in the art, and as described herein.

In some embodiments, the nucleic acids, vectors and cells embodied herein are targeted specifically to cells capable of expressing an MHC class II molecule. Targeted delivery to APCs, their stem cells or other precursor cell types can be achieved by receptor-mediated gene transfer using delivery vehicles comprising the following examples of targeting ligands: (a) for hemopoietic stem cells: anti-CD34 monoclonal antibody, or the Stem cell factor (c-Kit or CD117), or flk-2 ligand (human homolog STK-1); (b) for monocyte/macrophage/dendritic cell precursors: anti-CD33 monoclonal antibody; (c) for differentiated macrophage/dendritic cells: glycosylated DNA binding peptides carrying mannose groups may be used to target to specific receptors, for example the mannose receptor; and (d) for MHC class II bearing cells: an antibody that is specific for the constant region of MHC class II proteins or a ligand that binds MHC class II, for example soluble CD4; for example, one subset of MHC class bearing cells, B lymphocytes, may be targeted using soluble CD4 or using antibodies to or ligands for CD80, CD19, or CD22; for endothelial cells, γ-interferon and the vascular endothelial growth factor (VEGF) receptors; and (e) for APCs or T cells ligands for or antibodies to co-stimulatory molecules such as B7-1, B7-2 or CD28, CTLA-4, respectively. These targeting ligands may play a dual role which involves increasing co-stimulatory signals to the APC; and thus increasing its activation, in addition to their targeting function.

In other embodiments, DNA regulatory elements are used which lead to expression in APCs, their stem cells or other precursor cell types as a means of specifically targeting these cells.

In one embodiment, the cells, nucleic acids, vectors and/or compositions embodied herein are administered to a subject having or predisposed to neoplasia. In some embodiments, such use is in order to prevent, relieve, treat, ameliorate, prolong remission, suppress reactivation, etc. of neoplasia in a subject.

In one embodiment, the term “neoplasia” encompasses the process whereby one or more cells of an individual exhibiting abnormal growth characteristic. In one embodiment, such a process may comprise progression to the presence of a mass of proliferating cells in the individual. In another embodiment, neoplasia may refer to a very early stage in that only relatively few abnormal cell divisions have occurred. In one embodiment, an individual's predisposition to the development of a neoplasm is considered. Without limiting the present invention in any way, increased levels of or expression profiles of biomarkers in an individual who has not undergone the onset of neoplasia, may be indicative of that individual's predisposition to developing neoplasia.

In one embodiment, the term “predisposed to having neoplasia” refers to an individual with a higher risk factor or likelihood for developing neoplasia, such as, for example, an individual with a family history of neoplasia, or in another embodiment, an individual expressing genes associated with particular cancers, such as, for example, the so-called breast cancer genes, as described, for example, in U.S. Patent Application Publication Number 2004001852.

Cancer is a disease that involves the uncontrolled growth (i.e., division) of cells. Some of the known mechanisms which contribute to the uncontrolled proliferation of cancer cells include growth factor independence, failure to detect genomic mutation, and inappropriate cell signaling. The ability of cancer cells to ignore normal growth controls may result in an increased rate of proliferation. Although the causes of cancer have not been firmly established, there are some factors known to contribute, or at least predispose a subject, to cancer. Such factors include particular genetic mutations (e.g., BRCA gene mutation for breast cancer, APC for colon cancer), exposure to suspected cancer-causing agents, or carcinogens (e.g., asbestos, UV radiation) and familial disposition for particular cancers such as breast cancer. In some embodiments, neoplastic, hyperplastic or preneoplastic cells for use in the methods and/or compositions embodied herein may be obtained from individuals, or cell lines, exhibiting these phenomenon.

A subject having a cancer, in one embodiment, is a subject that has detectable cancerous cells.

A subject at risk of developing a cancer is one who has a higher than normal probability of developing cancer. These subjects include, for instance, subjects having a genetic abnormality that has been demonstrated to be associated with a higher likelihood of developing a cancer, subjects having a familial disposition to cancer, subjects exposed to cancer causing agents (i.e., carcinogens) such as tobacco, asbestos, or other chemical toxins, and subjects previously treated for cancer and in apparent remission.

In some embodiments, the neoplastic, preneoplastic or hyperplastic cells for use in the methods and/or compositions embodied herein, will express a cancer-associated antigen, in one embodiment, preferentially, or in another embodiment, at a greater concentration, or in another embodiment, in a particular form.

The tumor cells for use in the methods and compositions embodied herein can be prepared from virtually any type of tumor, as described herein.

In one embodiment, a composition is provided comprising a cell capable of expressing the major histocompatibility complex (MHC) class II protein, and a nucleic acid encoding a peptide or protein of interest fused in frame to a nucleic acid encoding the autophagosomal LC3 protein, or functional fragment thereof, or a vector comprising the same.

In some embodiments cells are administered to a subject at a concentration ranging may be from about 10×10⁴ to 1×10⁸, or in another embodiment 1×10⁶ to about 25×10⁶, or in another embodiment, from about 2.5×10⁶ to about 7.5×10⁶. In some embodiments, the cells are suspended in a pharmaceutically acceptable carrier or diluent, such as, but not limited to, Hank's solution (HMS), saline, phosphate-buffered saline, and water. In another embodiment, the tumor cells are at a concentration of from about 5×10⁴ to about 5×10⁶ cells, for example; 5×10⁴, 5×10⁵, or 5×10⁶ tumor cells.

In another embodiment, the solution in which the cells may be placed is in medium is which is serum-free, which may be, in another embodiment, commercially available, such as, for example, animal protein-free base media such as X-VIVO 10™ or X-VIVO 15™ (BioWhittaker, Walkersville, Md.), Hematopoietic Stem Cell-SFM media (GibcoBRL, Grand Island, N.Y.) or any formulation which promotes or sustains cell viability. Serum-free media used, may, in another emodiment, be as those described in the following patent documents: WO 95/00632; U.S. Pat. No. 5,405,772; PCT US94/09622. The serum-free base medium may, in another embodiment, contain clinical grade bovine serum albumin, which may be, in another embodiment, at a concentration of about 0.5-5%, or, in another embodiment, about 1.0% (w/v). Clinical grade albumin derived from human serum, such as Buminate® (Baxter Hyland, Glendale, Calif.), may be used, in another embodiment.

In another embodiment, the cells may be separated via affinity-based separation methods. Techniques for affinity separation may include, in other embodiments, magnetic separation, using antibody-coated magnetic beads, affinity chromatography, cytotoxic agents joined to a monoclonal antibody or use in conjunction with a monoclonal antibody, for example, complement and cytotoxins, and “panning” with an antibody attached to a solid matrix, such as a plate, or any other convenient technique. In other embodiment, separation techniques may also include the use of fluorescence activated cell sorters, which can have varying degrees of sophistication, such as multiple color channels, low angle and obtuse light scattering detecting channels, impedance channels, etc. It is to be understood that any technique, which enables separation of the cells of or for use herein may be employed, and is to be considered as part embodied herein.

In another embodiment, the affinity reagents employed in the separation methods may be specific receptors or ligands for the cell surface molecules indicated hereinabove.

In another embodiment, the antibodies utilized herein may be conjugated to a label, which may, in another embodiment, be used for separation. Labels may include, in other embodiments, magnetic beads, which allow for direct separation, biotin, which may be removed with avidin or streptavidin bound to, for example, a support, fluorochromes, which may be used with a fluorescence activated cell sorter, or the like, to allow for ease of separation, and others, as is well known in the art. Fluorochromes may include, in one embodiment, phycobiliproteins, such as, for example, phycoerythrin, allophycocyanins, fluorescein, Texas red, or combinations thereof.

In one embodiment, cell separations utilizing antibodies will entail the addition of an antibody to a suspension of cells, for a period of time sufficient to bind the available cell surface antigens. The incubation may be for a varied period of time, such as in one embodiment, for 5 minutes, or in another embodiment, 15 minutes, or in another embodiment, 30 minutes. Any length of time which results in specific labeling with the antibody, with minimal non-specific binding is to be considered envisioned for this aspect.

In another embodiment, the staining intensity of the cells can be monitored by flow cytometry, where lasers detect the quantitative levels of fluorochrome (which is proportional to the amount of cell surface antigen bound by the antibodies). Flow cytometry, or FACS, can also be used, in another embodiment, to separate cell populations based on the intensity of antibody staining, as well as other parameters such as cell size and light scatter.

The separated cells may be collected in any appropriate medium that maintains cell viability, and may, in another embodiment, comprise a cushion of serum at the bottom of the collection tube.

In another embodiment, the culture containing the cells for use herein may contain other cytokines or growth factors to which the cells are responsive. In one embodiment, the cytokines or growth factors promote survival, growth, function, or a combination thereof. In another embodiment, the culture containing the cells of or for use herein may contain polypeptides and non-polypeptide factors.

In other embodiments, the methods and/or compositions embodied herein may comprise known cancer medicaments, such as those known to prime the immune system to attack the neoplastic, preneoplastic or hyperplastic cells. In other embodiments, methods and/or compositions embodied herein may comprise known cancer medicaments such as angiogenesis inhibitors, which function by attacking the blood supply of solid tumors. Since the most malignant cancers are able to metastasize (i.e., exist the primary tumor site and seed a distal tissue, thereby forming a secondary tumor), medicaments that impede this metastasis are also useful in the treatment of cancer. Angiogenic mediators may include basic FGF, VEGF, angiopoietins, angiostatin, endostatin, TNF-α, TNP-470, thrombospondin-1, platelet factor 4, CAI, and certain members of the integrin family of proteins, and thus, in some embodiments, angiogenesis inhibitors may specifically targeted to prevent the activity or proper functioning of such molecules. In one embodiment, the inhibitor may comprise a metalloproteinase inhibitor, which inhibits the enzymes used by the cancer cells to exist the primary tumor site and extravasate into another tissue.

In other embodiments, the methods embodied herein are for use in preventing neoplasia, or in another embodiment, preventing metastasis in a subject. Tumor metastasis involves the spread of tumor cells primarily via the vasculature to remote sites in the body. In one embodiment, the term “metastases” shall mean tumor cells located at sites discontinuous with the original tumor, usually through lymphatic and/or hematogenous spread of tumor cells. In one embodiment, the term metastasis refers to the invasion and migration of tumor cells away from the primary tumor site. A metastasis is, in some embodiments, a region of cancer cells, distinct from the primary tumor location resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

The terms “prevent” and “preventing” as used herein with respect to metastasis refer to inhibiting completely or partially the metastasis of a cancer or tumor cell, as well as inhibiting any increase in the metastatic ability of a cancer or tumor cell.

The invasion and metastasis of cancer is a complex process which involves changes in cell adhesion properties which allow a transformed cell to invade and migrate through the extracellular matrix (ECM) and acquire anchorage-independent growth properties. Liotta, L. A., et al., Cell 64:327-336 (1991). Some of these changes occur at focal adhesions, which are cell/ECM contact points containing membrane-associated, cytoskeletal, and intracellular signaling molecules. Metastatic disease occurs when the disseminated foci of tumor cells seed a tissue which supports their growth and propagation, and this secondary spread of tumor cells is responsible for the morbidity and mortality associated with the majority of cancers.

In some embodiments, the methods embodied herein and/or compositions embodied herein specifically make use of cells at the initiation of, or during metastasis, as a means of treating, or in another embodiment, preventing, or in another embodiment, delaying the onset of, or in another embodiment, halting the progression of metastasis.

In some embodiments, the methods and/or compositions embodied herein provide for a long-lived systemic immune response, and may therefore be effective not only against the primary tumor, but also against metastatic cells sharing tumor antigen with the primary tumor. In some embodiments, the methods and/or compositions embodied herein may be useful in combating multiple types of tumors, which may be somewhat related in terms of, for example, the antigens expressed or downregulated in such tumors and represent embodiments herein.

In one embodiment, the methods and/or compositions embodied herein are for the treatment of cancer. In one embodiment, the term “treatment” refers to intervention in an attempt to alter the natural course of the individual or cell being treated, and may be performed either for prophylaxis or during the course of clinical pathology. Desirable effects include preventing occurrence or recurrence of disease, alleviation of symptoms, diminishment of any direct or indirect pathological consequences of the disease, preventing metastasis, lowering the rate of disease progression, amelioration or palliation of the disease state, and remission or improved prognosis.

In some embodiments, a method is provided for downmodulating, suppressing or tolerizing an immune response in a subject to a peptide or protein of interest. Such methods are useful for preventing or diminishing transplant rejection and autoimmune diseases, by way of non-limiting examples. In transplant rejection (host-vs.-graft disease), the antigen against which the immune response is desirably downmodulated comprises a peptide or protein of the graft (transplant). In graft-vs.-host disease, where immune cells in the graft attack the host, the antigen against which the immune response is desirably downmodulated comprises a peptide or protein of the transplant recipient, or host. In other embodiments, autoimmunity can be treated by the methods generally described herein wherein the peptide or protein of interest is a self, or host, antigen. In the practice of this aspect, immature dendritic cells are contacted with a nucleic acid encoding a peptide or protein of interest fused in frame to a nucleic acid encoding an autophagosomal targeting protein, or a functional fragment thereof. Contacting can be ex vivo, or in vivo, the latter typically by targeting a vector to immature dendritic cells. The autophagosomal targeting protein or functional fragment thereof targets said peptide or protein of interest to an autophagosome, and said peptide, or a fragment of said protein of interest is displayed on the surface of said immature dendritic cell in the context of a major histocompatibility complex (MHC) class II molecule. This results in a downmodulation of the immune response to the peptide or protein of interest through the process of T cell deletion or anergy. In the steady state, dendritic cells are in an immature state and not fully differentiated to carry out their known roles as inducers of immunity. Nevertheless, immature dendritic cells continuously circulate through tissues and into lymphoid organs, capturing self antigens as well as innocuous environmental proteins. Recent experiments (as reviewed by Steinman and Nussenzweig, 2002, Proc. Nat. Acad. Sci. U.S.A. 99, 351-358) have provided direct evidence that antigen-loaded immature dendritic cells silence T cells either by deleting them or by expanding regulatory T cells.

In one embodiment the autophagosomal targeting protein is an LC3 protein. In another embodiment, the nucleic acid comprises a sequence homologous to, or corresponding to SEQ ID NO: 1. As noted above, wherein the method is used to prevent or diminish transplant rejection or graft-vs.-host disease in the subject, the peptide or protein of interest is a graft antigen, derived from the tissue or organ transplanted; or a host (self) antigen. Where the method is used to treat autoimmunity in the subject, the peptide or protein of interest is a self antigen. Non-limiting examples of autoimmune diseases amenable to treatment include diabetes mellitus type 1 (IDDM), systemic lupus erythematosus (SLE), Sjögren's syndrome, Hashimoto's thyroiditis, Graves' disease, and rheumatoid arthritis (RA).

The “pathology” associated with a disease condition is anything that compromises the well-being, normal physiology, or quality of life of the affected individual. This may involve (but is not limited to) destructive invasion of affected tissues into previously unaffected areas, growth at the expense of normal tissue function, irregular or suppressed biological activity, aggravation or suppression of an inflammatory or immunological response, increased susceptibility to other pathogenic organisms or agents, and undesirable clinical symptoms such as pain, fever, nausea, fatigue, mood alterations, and such other features as may be determined by an attending physician.

In some embodiments, the nucleic acids, vectors, cells, methods and/or compositions embodied herein provide for prevention, suppression, treatment, amelioration of symptoms, etc., of any infection, as described herein. In some embodiments, the antigens are disease specific, or in another embodiment, multiple antigens from multiple infections/diseases are utilized as a pan vaccine strategy, as will be appreciated by one skilled in the art.

In some embodiments, the nucleic acids, vectors, cells embodied herein are provided to the subject in an effective amount, which in one embodiment, refers to an amount sufficient to effect a beneficial or desired clinical result, particularly the generation of an immune response, or noticeable improvement in clinical condition. An immunogenic amount is an amount sufficient in the subject group being treated (either diseased or not) to elicit a desired immunological response. In terms of clinical response for subjects with disease, an effective amount is amount sufficient to palliate, ameliorate, stabilize, reverse or slow progression of the disease, or otherwise reduce pathological consequences of the disease. An effective amount may be given in single or divided doses. It is to be understood that the methods and/or compositions embodied herein may provide an immunogenic or therapeutically effective amount, both of which are to be considered embodied herein.

In some embodiments, the treatment can be ascertained via standard protocols for monitoring disease progression, for example, in the case of subjects with tumors, such monitoring may be effected, for example, via the use of magnetic resonance imaging (MRI), radioscintigraphy with a suitable imaging agent, monitoring of circulating tumor marker antigens, the subject's clinical response, or a combination thereof. For example, and in one embodiment, an appropriate clinical marker is serum CA-125 for the monitoring of advanced ovarian cancer. Hempling et al. (1993) J. Surg. Oncol. 54:38-44.

The administration of the compositions and/or cells according to the methods embodied herein may be conducted as appropriate, for example on a monthly, semimonthly, or in another embodiment, on a weekly basis, until the desired effect is achieved. Thereafter, and particularly when the immunological or clinical benefit appears to subside, additional booster or maintenance regimens may be undertaken, and designed as appropriate, as will be appreciated by one skilled in the art.

When multiple doses of a cellular vaccine are given to the same patient, some attention should be paid to the possibility that the allogeneic lymphocytes in the vaccine may generate an anti-allotype response. The use of a mixture of allogeneic cells from a plurality of donors, and the use of different allogeneic cell populations in each dose, are both strategies that can help minimize the occurrence of an anti-allotype response.

During the course of therapy, the subject is evaluated on a regular basis for side effects at the injection site, or general side effects such as a febrile response. Side effects are managed with appropriate supportive clinical care.

In some embodiments, gene delivery systems are provided, which in some embodiments, make use of viral vectors, that contain an autophagosomal targeting protein, which in one embodiment is LC3, coupled to viral, bacterial or tumor antigens. Vaccination with these systems, in some embodiments, boosts CD4+ T cell immunity against the targeted antigens without the necessity for the fusion protein to be expressed in every infected or tumor cell.

In some embodiments, methods are provided which employ vector administration for active immunization, ex vivo infection for adoptive transfer of for example dendritic cells for active immunization, ex vivo stimulation of CD4+ T cells for passive immunization, or a combination thereof, via targeting antigen for MHC class II presentation as described herein. In one embodiment, non-diseased antigen presenting cells are used according to the methods embodied herein, for ex vivo transfection with an LC3-antigen fusion protein. In some embodiments, according to these aspects embodied herein, monocytes, macrophages, dendritic cells, B cells and epithelial cells, are used.

In another embodiment, the composition may further comprise an adjuvant, such as, for example, technic acids from gram negative bacteria, such as LTA, RTA, GTA, and their synthetic counterparts, hemocyanins and hemoerythrins, such as KLH, chitin or chitosan. In another embodiment, the adjuvant may comprise muramyl dipeptide (MDP) and tripeptide peptidoglycans and their derivatives, such as threonyl-NDP, fatty acid derivatives, such as MTPPE, and the derivatives described in U.S. Pat. No. 4,950,645, incorporated herein by reference. BCG, BCG-cell wall skeleton (CWS) and trehalose monomycolate and dimycolate (U.S. Pat. Nos. 4,579,945 and 4,520,019, each incorporated herein by reference) may also be used as adjuvants herein, either singly or in combinations of two or three agents, or in combination with monophosphoryl lipid A (MPL) (see for example as described by Johnson et al. (1990), Grabarek et al. (1990), Baker et al. (1992; 1994); Tanamoto et al. (1994a; b; 1995); Brade et al. (1993) and U.S. Pat. No. 4,987,237). Amphipathic and surface active agents, such as QS21, and nonionic block copolymer surfactant form yet another group of preferred adjuvants. Although useful in all aspects herein, these adjuvants may find particular utility in compositions for use in generating or enhancing the immune response against intracellular antigens, including intracellular tumor antigens.

In another embodiment, the compositions embodied herein may include bulk drug compositions useful in the manufacture of pharmaceutical compositions (e.g., impure or non-sterile compositions) and pharmaceutical compositions (i.e., compositions that are suitable for administration to a subject or patient), which can be used in the preparation of unit dosage forms. Such compositions comprise a prophylactically or therapeutically effective amount of a prophylactic and/or therapeutic agent disclosed herein or a combination of those agents and a pharmaceutically acceptable carrier.

In one embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers, in another embodiment, to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is another carrier, which, in another embodiment, is used when the pharmaceutical composition is administered intravenously. Saline solutions and aqueous dextrose and glycerol solutions can also be employed as liquid carriers, in other embodiments, including injectable solutions. Suitable pharmaceutical excipients may include, in other embodiments, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like.

The compositions embodied herein can be formulated as neutral or salt forms. Pharmaceutically acceptable salts include, but are not limited to those formed with anions such as those derived from hydrochloric, phosphoric, acetic, oxalic, tartaric acids, etc., and those formed with captions such as those derived from sodium, potassium, ammonium, calcium, ferric hydroxides, isopropylamine, triethylamine, 2-ethylamino ethanol, histidine, procaine, etc.

According to these aspects, and in one embodiment, the compositions embodied herein and/or for use in the methods embodied herein may be at a dose and schedule, which will vary depending on the age, health, sex, size and weight of the subject to which it will be administered. These parameters can be determined for each system by well-established procedures and analysis, e.g., in phase I, II and III clinical trials, or other means, as will be appreciated by one skilled in the art.

For administration, the cells, nucleic acids and/or vectors embodied herein can be combined with a pharmaceutically acceptable carrier such as a suitable liquid vehicle or excipient and an optional auxiliary additive or additives. The liquid vehicles and excipients are conventional and are commercially available. Illustrative thereof are distilled water, physiological saline, aqueous solutions of dextrose and the like.

Suitable formulations for parenteral, topical, mucosal, for example, oral, intranasal, etc., or intraperitoneal administration, include aqueous solutions of active compounds in water-soluble or water-dispersible form. In addition, suspensions of the active compounds as appropriate oily injection suspensions may be administered. Suitable lipophilic solvents or vehicles include fatty oils for example, sesame oil, or synthetic fatty acid esters, for example, ethyl oleate or triglycerides. Aqueous injection suspensions may contain substances which increase the viscosity of the suspension, include for example, sodium carboxymethyl cellulose, sorbitol and/or dextran, optionally the suspension may also contain stabilizers. In other embodiments, the cells can be mixed with immune adjuvants well known in the art such as Freund's complete adjuvant, inorganic salts such as zinc chloride, calcium phosphate, aluminum hydroxide, aluminum phosphate, saponins, polymers, lipids or lipid fractions (Lipid A, monophosphoryl lipid A), modified oligonucleotides, etc.

General procedures for the preparation and administration of pharmaceutical compositions are outlined in Remington's Pharmaceutical Sciences 18th Edition (1990), E. W. Martin ed., Mack Publishing Co., PA, and represent embodiments herein.

In addition to administration with conventional carriers, the cells, nucleic acids, vectors and/or other active ingredients may be administered by a variety of specialized delivery drug techniques which are known to those of skill in the art. The following examples are given for illustrative purposes only and are in no way intended to limit the invention.

### EXAMPLES

### Materials and Methods

### Cell Lines

HaCat (human keratinocyte cell line) was a gift from Rajiv Khanna, Brisbane, Australia. MDAMC (human breast carcinoma cell line) was a gift from Irene Joab, Paris, France. HeLa and 293 were purchased from ATCC. The EBV-transformed B lymphocyte cell line MS-LCL was generated by culturing PBMCs of a healthy donor with supernatant of the marmoset cell line B95-8 [Miller, G., et al. IARC Sci Publ (11, 395-408. (1975)] with RPMI-1640+20% FCS+2 mM glutamine+2 μg/ml gentamycin+1 μg/ml Cyclosporin A. The two EBV-positive Hodgkin's lymphoma cell lines RPMI6666 and L591 were purchased from ATCC and a gift from Martina Vockerodt and Dieter Kube, Gottingen, Germany, respectively. Mouse hybridomas IVAl2 (anti-human MHC class II) and w6/32 (anti-human MHC class I) were purchased from ATCC. Epithelial cell lines were routinely cultured in DMEM with 10% FCS, 2 mM glutamine, 110 μg/ml sodium pyruvate and 2 μg/ml gentamicin. B cell lines and hybridomas were maintained in RPMI-1640 with 10% FCS+glutamine+gentamicin.

### Monocyte and Dendritic Cell Preparation.

Leukocyte concentrates (buffy coats) from the New York Blood Center or blood donations from healthy lab donors served as sources of PBMCs and were isolated by density gradient centrifugation on Ficoll-Paque (Amersham-Pharmacia Biotech). Positive selection for CD14-positive monocytes/macrophages was performed using anti-CD14 MicroBeads from Miltenyi Biotec, Bergisch-Gladbach, Germany. Dendritic cells (DCs) were generated from CD14-positive cells, by plating 6.6×10⁵ CD14⁺ cells/ml into six-well plates with RPMI-1640+1% single-donor plasma+glutamine+gentamycin. Recombinant human IL-4 (rhIL-4, 500 U/ml) and rhGMCSF (1000 U/ml) were added on day 0, 2, and 4. On day 5, floating immature DCs were transferred to new plates at 3.3×10⁵ cells/ml and half of the medium was replaced with fresh medium containing LPS (100 ng/ml, Sigma, St. Louis, Mo.) or IL-1β (10 ng/ml), IL-6 (1000 U/ml), TNF-α (10 ng/ml) and PGE₂ (1 μg/ml) to mature dendritic cells. All cytokines were obtained from R&D Systems (Minneapolis, Minn.), Peprotech (Rocky Hill, N.J.), Berlex (Richmond, Calif.) or Sigma (St. Louis, Mo.).

### T Cell Clones.

The influenza A matrix protein 1 (MP1)-specific T cell clones 9.2, 9.26 and 10.9 were generated as previously described [Fonteneau, J. F. et al. J Immunol Methods 258, 111-26. (2001)]. Briefly, CD14-negative PBMCs isolated from whole blood of a lab donor (HLA-A*0201, -A*6801, -B*4402, -B*0702, -C*0501, -C*0702, -DRB1*1501, -DRB1*0401, -DRB5*01, -DRB4*01, -DQBI*0602 and -DQB1*0301) were stimulated with autologous mature DCs electroporated with in vitro transcribed Influenza A MP1-RNA, a gift from Irina Tcherepanova of Argos Therapeutics, Durham, N.C. (PBMC:DC ratio=30:1, medium: RPMI-1640 with 5% human serum+glutamine+gentamicin). DCs were electroporated with 10 μg RNA in Opti-MEM at 300V and 150 μF with a BioRad Gene Pulser plus Capacitance Extender (BioRad, Hercules, Calif.). On day 8 of PBMC/DC coculture, the stimulation was repeated and 10 U/ml IL-2 were added to enhance T cell survival. On day 21, the surviving cells were cloned by limiting dilution at 10, 1, or 0.3 cells/well and expanded in RPMI-1640+8% PHS+150 U/ml rhIL-2 (Chiron, Emeryville, Calif.)+1 μg/ml PHA-L (Sigma-Aldrich, St. Louis, Mo.)+glutamine+gentamicin. 10⁵ irradiated PBMCs/well and 10⁴ irradiated LCLs/well were added as feeder cells. On day 40, expanded cells were tested in split-well IFNγ ELISPOT assays for recognition of an MP1 peptide mix (64 15-mer peptides overlapping by 10 amino acids) and the HLA-A2 immunodominant epitope MP1₅₈₋₆₆ (GILGFVFTL). MP1-specific clones were tested for CD4/CD8 expression by staining with anti-CD4-PE and anti-CD8-PE (BD Biosciences Pharmingen, San Diego, Calif.) and subsequent analysis by flow cytometry. MP1-specific, homogenously CD4⁺ or CD8⁺ clones were expanded as described above and frozen into aliquots of 5×10⁶ cells/cryovial. All peptides were purchased from the Proteomics Resource Center of the Rockefeller University.

### Inhibitors and Recombinant Proteins.

Chloroquine (CQ) was purchased from Sigma, St. Louis, Mo., and used at 50 μM. Recombinant human IFNγ was purchased from ProSpec-Tany TechnoGene LTD, Israel and was used at 200 U/ml.

### LC3 Fusion Constructs and Generation of Stable Transfectants.

The cDNA of human MAP1LC3B sequence (NM_{—}022818) was cloned from a human B-LCL by RT-PCR with gene specific primers into the mammalian expression vector pEGFP-C2 (Clontech, Mountain View, Calif.). To generate HeLa and 293 cell lines stably expressing GFP-LC3, the EGFP-LC3 construct was transiently transfected into cell lines using lipofectamine 2000 (Invitrogen, Carlsbad, Calif.) and cells were subsequently cultured in the presence of 500 μg/ml G418. The cDNA of Influenza A/WSN/33 matrix protein 1 (MP1) was PCR-amplified from the pCAGGS/MCS-MP1 vector, a gift from Peter Palese, Mount Sinai School of Medicine, New York, with or without a stop codon at the 3′ end. The PCR products then were inserted into the pEGFP-LC3 vector in place of the EGFP sequence to obtain MP1-LC3 fusion constructs. For lentiviral constructs, the EGFP-LC3, MP1-LC3 or MP1Stop-LC3 sequences were subcloned into the lentiviral vector pHR-SIN-CSGWΔNotI, a gift from Jeremy Luban, Columbia University, New York. For production of lentiviral particles, lentiviral vectors were co-transfected with the helper plasmids pCMVΔR8.91 and pMDG into 293T cells by calcium phosphate transfection. Culture supernatants containing recombinant viral particles were harvested on day 1, 2 and 3 after transfection, filtered through a 0.45 μm filter and frozen at −80° C. HaCat and MDAMC cell lines stably expressing GFP-LC3, MP1-LC3 or MP1 were generated by lentiviral infection using MOIs of 10-40.

### Antisera.

The LC3 antiserum was generated by immunizing two rabbits with the N-terminal peptide LC3₁₋₁₅ (MPSEKTFKQRRTFEQR; SEQ ID NO:4) conjugated to KLH carrier protein (Cocalico Biologicals, Reamstown, Pa.). Animals were boosted 5 times (2, 3, 7, 11 and 15 weeks after initial inoculation) and then sacrificed to obtain terminal bleeds. Antiserum collected from one rabbit showed good LC3 reactivity by ELISA and Western blot and was used for Western blots at a dilution of 1:2000. Influenza MP1-specific rabbit antiserum was a gift from Ari Helenius, Zurich, Switzerland.

Knockdown of atg12

The following 21-nt siRNA oligos were used:

| | |
|---|---|
| Atg12 sense: | |
| 5′-UCAACUUGCUACUACAUGAUdT; | (SEQ ID NO: 5) |

Atg12 antisense: 5′-UCAUGUAGUAGCAAGUUGAUdT (SEQ ID NO:6; nt. 687-705 of NM_{—}004707). As a control, lamin A/Cspecific siRNA from Dharmacon (Lamin sense: 5′-CUGGACUUCCAGAAGAACAdTdT (SEQ ID NO:7); Lamin antisense: 5′-UGUUCUUCUGGAAGUCCAGdTdT (SEQ ID NO:8) or firefly luciferase-specific siRNA (GL2 sense: 50-CGUACGCGGAAUACUUCGAdTdT; SEQ ID NO:9; GL2 antisense: 5′-UCGAAGUAUUCCGCGUACGdTdT; SEQ ID NO:10) was used. siRNA duplexes were delivered by transfection with lipofectamine 2000 (Invitrogen) at 30 pmol siRNA+1.5 ml lipofectamine/well in a 24-well format, and effect of knockdown was analyzed after 2-3 days.

### Lysate Preparation and Immunoblotting.

Cells were lysed in ice cold lysis buffer (50 mM Tris-HCl pH 8.0, 140 mM NaCl, 1 mM DTT, 1.5 mM MgCl₂, 0.5% NP-40 with Complete protease inhibitor cocktail, Roche, Indianapolis, Ind.) for 5 mim on ice (about 10⁶ cells/100 μl). Whole cells and cell debris were pelleted by low speed centrifugation (400 g, 3 mil) and cleared supernatants were transferred to a new tube. Protein concentration was determined by BCA protein assay (Pierce, Rockford, Ill.). Samples were boiled for 5 min in the presence of 4×SDS-PAGE-loading buffer (250 mM Tris-HCl pH 6.8, 40% glycerol, 8% SDS, 0.57 M β-mercaptoethanol, 0.12% bromophenol blue). Equal amounts of protein were run on 11 or 12% SDS-PAGE gels and transferred onto a PVDF membrane (Hybond-P, Amersham Biosciences, UK). Primary antibodies were visualized with HRP-conjugated goat anti-rabbit IgG (Biorad, Hercules; CA) and the ECLplus detection system (Amersham Biosciences, UK). As a loading control, blots were reprobed with an anti-β-actin antibody (clone AC-40, Sigma, St. Louis, Mo.) and HRP-conjugated goat anti-mouse IgG (Biorad, Hercules, Calif.).

### Immunocytochemistry and Confocal Microscopy.

Epithelial cells were plated onto microscopy cover glasses in a 24 well plate and cultured overnight at 37° C. Cells were washed 1× in PBS and fixed in 3% paraformaldehyde in PBS for 15 minutes at room temperature (RT). Cells were washed 1× in PBS and permeabilized in 0.1% Triton X-100 in PBS for 5 min at RT. After another rinse in PBS, cells were blocked for 30 min in blocking buffer (from Perkin Elmer's TSA kit)+0.1%® saponin. Primary antibody was added in blocking buffer+0.1% saponin+5% normal serum (goat or donkey, depending on the secondary antibody) for 30-60 min at RT (primary antibodies: Anti-MHC class I and II antibodies (hybridoma supernatants IVA12 and w6/32 hybridomas, ATCC), anti-HLA-DM (clone MaP-DM1, BD Biosciences Pharmingen, San Diego, Calif.), anti-LAMP-2 (clone H₄B4, Southern Biotechnology Associates, Birmingham, Ala.), anti-EEA1 (Santa Cruz Biotech, Santa Cruz, Calif.) and anti-transferrin receptor (clone DF 1513, Sigma, St. Louis, Mo.)). Cells were washed 3×5 min in PBS+0.1% saponin and were incubated with secondary antibody in blocking buffer+0.1% saponin+5% normal goat or donkey serum for 30 min in the dark (Secondary antibodies: Rhodamine-Red™-X-(RRX)-conjugated donkey anti-mouse IgG and RRX-conjugated donkey anti-goat IgG (Jackson ImmunoResearch, West Pine, Pa.) and Alexa546-conjugated goat-anti-rabbit IgG (Invitrogen-Molecular Probes, Carlsbad, Calif.)). Cells were then incubated with DAPI nucleic acid stain (0.5 μg/ml, Molecular Probes) for 1 min and subsequently washed 3×5 min in PBS+0.1% saponin and 1× in PBS. Finally, cover glasses were mounted onto microscope slides using Prolong Gold antifade reagent (Invitrogen-Molecular Probes, Carlsbad, Calif.) and slides were allowed to dry in the dark. Cells were analyzed using an inverted LSM 510 laser scanning confocal microscope (Zeiss Axiovert to 200) with a 63x/1.4 N.A. oil immersion lens using a 405 nm diode laser and an argon laser (488 nm and 543 nm laser lines) and a pinhole diameter of 1 Airy unit. Pictures were taken with the LSM 510 confocal software (Zeiss). Colocalization of markers was quantified using the profile and histogram tools of the LSM 510 software. In the profile analysis, the number of double-positive vesicles compared to the total number of red vesicles was determined in 10-15 double-positive cells/condition. In the histogram analysis, the number of colocalized pixels compared to the total number of red channel pixels above background was determined in 10-15 double-positive cells/condition (background threshold was determined for each cell by the LSM510 algorithm).

### Electron Microscopy and Immunolabeling of Cryosections.

MDAMC cells stably transfected with GFP-LC3 were fixed for 1 h at RT with 4% paraformaldehyde (PFA, Electron Microscopy Sciences) in 0.25 M Hepes, pH 7.4, followed by overnight fixation at 4° C. in 8% PFA/Hepes. Cells were washed 1× in PBS, quenched with 0.1 M NH₄Cl in PBS for 10 min, scraped into 1% gelatin in PBS and then embedded in 5% gelatin in PBS. Small pieces of the gelatin pellets were infiltrated overnight at 4° C. with 2.3 M sucrose in PBS, mounted onto cryospecimen pins and frozen in liquid nitrogen. Ultrathin sections (80 nm) were cut using a Leica ultracut ultramicrotome with an FCS cryoattachment at −108° C. and collected on formvar- and carbon-coated nickel grids using a 1:1 mixture of 2% methyl cellulose (25 centipoises; Sigma-Aldrich) and 2.3 M sucrose in PBS. After quenching with 0.1 M NH₄Cl in PBS for 10 min, the grids were incubated for 20 min in a solution of 1% fish skin gelatin (FSG, Sigma-Aldrich) in PBS. They were then labeled with rabbit anti-HLA-DR antiserum C6861 (a gift from Peter Cresswell, Yale University, New Haven, Conn.) in PBS-FSH for 30 min at RT, washed 4× in PBS and then incubated with 10 or 15 nm protein A-gold (purchased from the Department of Cell Biology, University of Utrecht, Netherlands). After 4 more washes in PBS, grids were fixed in 1% glutaraldehyde in PBS for 5 min at RT, washed 5× in PBS and again quenched with 0.1 M NH₄Cl in PBS for 10 min. The same labeling procedure was repeated with a rabbit anti-GFP antibody (Invitrogen-Molecular Probes, Carlsbad, Calif.) and after final fixation in 1% glutaraldehyde, grids were washed 8× in HPLC-grade water. Sections were infiltrated for 10 min on ice with a mixture of 1.8% methylcellulose and 0.5% uranyl acetate (Electron Microscopy Sciences, Hatfield, Pa.), washed 3× in 0.5% uranyl acetate/1.8% methylcellulose and air-dried. Samples were analyzed in a Tecnai 12 Biotwin (FEI) microscope and pictures were taken using Kodak 4489 film.

### T Cell Assay.

MP1- and MP-LC3-expressing target cells were treated with 200 U/ml IFNγ for 24 h to upregulate MHC class II expression. Cells were washed 3× in DMEM to remove IFNγ, detached with to Trypsin-EDTA and resuspended in RPMI-1640 with 5% PHS+glutamine+gentamicin (5% PHS medium). T cell clones were washed 1× in 5% PHS medium and plated into a round bottom 96 well plate at 10⁵ T cells/well. Target cells were added at E:T ratios of 2, 5 and 12.5, i.e. 5×10⁴, 2×10⁴ and 8×10³ targets/well.

### IFNγELISA.

IFNγ in culture supernatants was measured using the human IFNγ ELISA from Mabtech Inc., Mariemont, Ohio. Briefly, 96-well Nunc-Immuno™ MaxiSorp plates (Nalge Nunc Intl., Rochester, N.Y.) were coated with primary anti-IFNγ antibody 1-D1K (Mabtech) overnight and blocked with 1% BSA in PBS for 1 h. Culture supernatants from CD4⁺ T cell clones were diluted 1:2 and supernatant from the CD8⁺T cell clone were diluted 1:20 in PBS/0.1% BSA/0.05%® Tween-20. Diluted supernatants were added to the plate for 2 h, followed by incubation with biotinylated anti-IFNγ antibody 7-B6-1 (Mabtech) and Streptavidin-HRP (Mabtech) for 1 h each. IFNγ was detected using the peroxidase substrate TMB (Sigma, St. Louis, Mo.). Recombinant human IFNγ (Mabtech) diluted in PBS/0.1% BSA/0.05% Tween-20 (2,000 to 30 pg/ml) was used as a standard.

### Flow Cytometry.

MHC class II surface levels on epithelial cells were measured by staining cells with IVA12 hybridoma supernatant and AlexaFluor488-conjugated rabbit α-mouse IgG (Invitrogen-Molecular Probes, Carlsbad, Calif.). Cells were analyzed on a FACScalibur instrument (Becton-Dickinson).

### Statistics.

Paired or homocedastic, one-tailed student's T test statistics were applied where indicated.

### Example 1

### Autophagosomes are Constitutively Turned Over by Lysosomal Proteases in Human Epithelial Cells Lines

In order to test whether MHC class II-positive human cells exhibit constitutive autophagy, the level of autophagy in human epithelial cell lines was determined. Epithelial cells readily up-regulate MHC class II molecules in response to inflammatory cytokines both in vitro and in vivo. It was thus of interest to determine whether these cell lines might rely on endogenous degradation pathways, such as autophagy, to generate peptide ligands for their MHC class II molecules. To quantify autophagy, the specific autophagosome marker Atg8/LC3 was used. LC3 is a ubiquitin-like protein that is covalently coupled via its C-terminus to a phospholipid in the newly forming inner and outer autophagosomal membranes and thus is specifically incorporated into autophagosomes. Autophagosomes are short-lived (t_{1/2}=8 min) and rapidly fuse with endosomes or lysosomes to form so-called amphisomes or autolysosomes. In these fusion compartments, intraluminal LC3 is rapidly degraded by lysosomal proteases. The more autophagosomes that are formed, the more LC3 is degraded in autolysosomes and therefore, lysosomal turnover of LC3 is a good measure for autophagic activity.

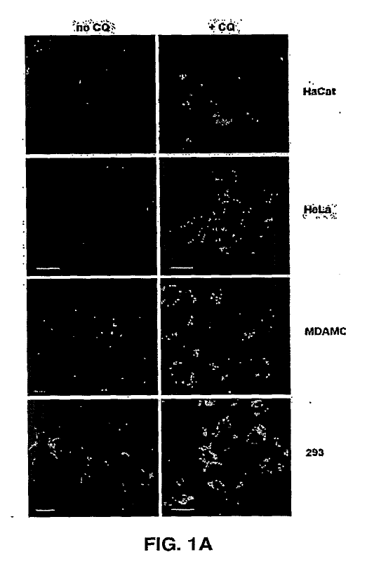

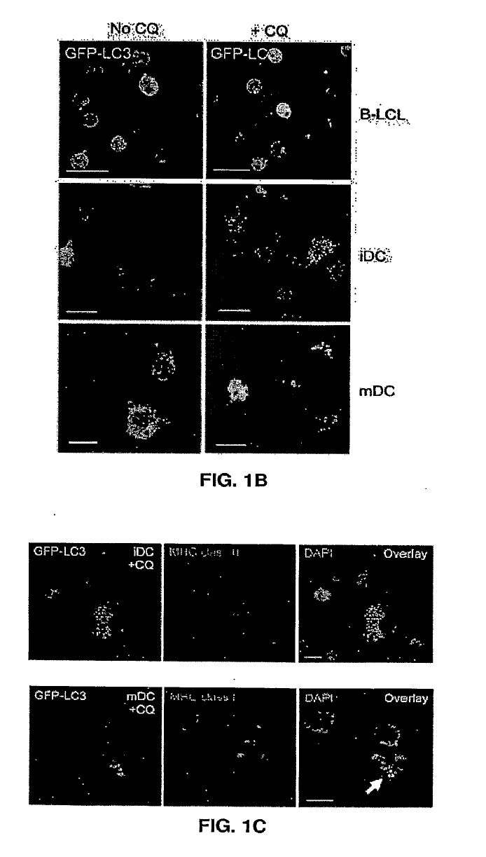

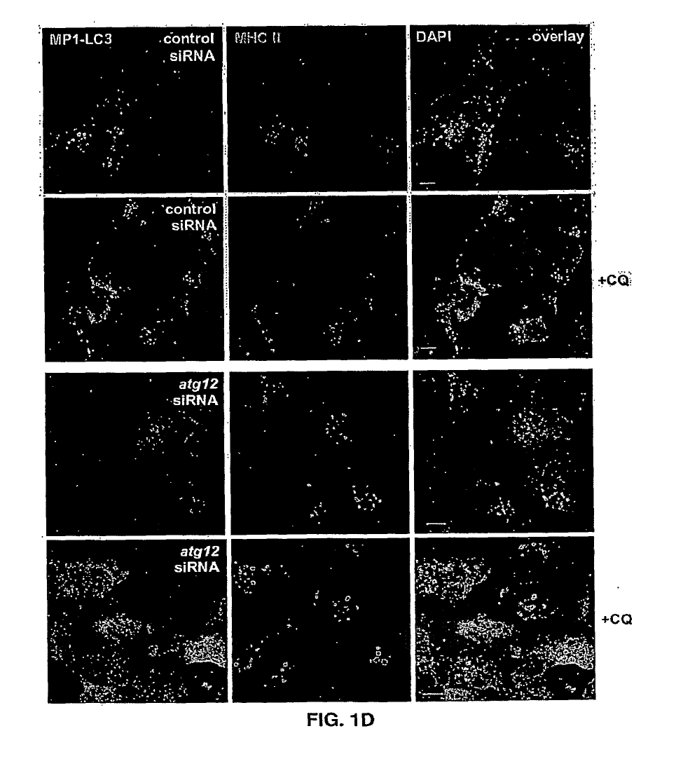

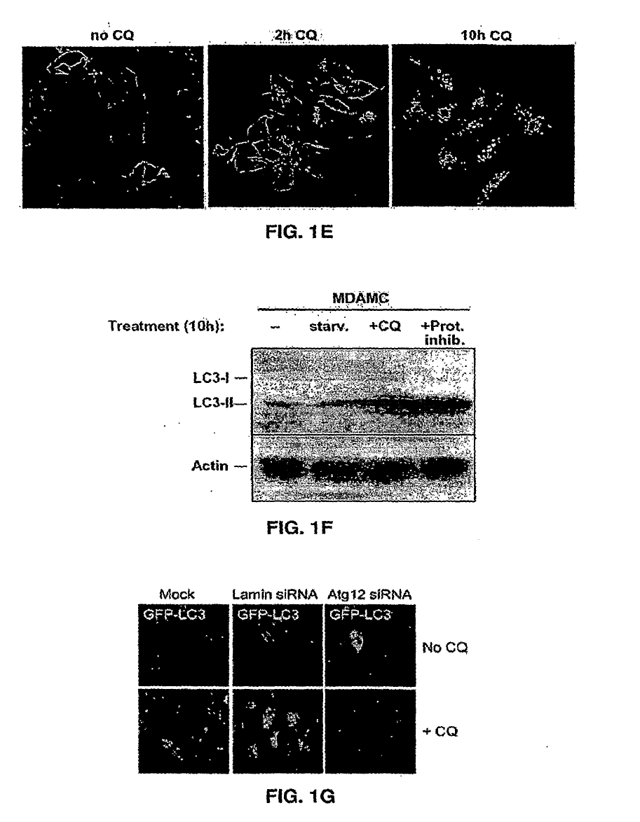

To visualize the lysosomal turnover of LC3 in human epithelial cells by fluorescence microscopy, cell lines derived from different organs [HaCat (skin), HeLa (cervix), MDAMC (breast), 293 (kidney)] were transfected with a GFP-LC3 fusion construct. GFP-LC3 reporter constructs have been used previously to visualize autophagosomes in transgenic mice and cultured cells, and it has been shown that overexpression of GFP-LC3 does not alter the autophagic activity. GFP-LC3 transfected cell lines were treated with the lysosomal acidification inhibitor chloroquine (CQ) to block lysosomal proteolysis and thus visualize the accumulation of GFP-LC3 in autolysosomes. In all cell lines analyzed in this study, GFP-LC3 strongly accumulated in cytosolic vesicles after 10 hours of CQ-treatment (FIG. 1A), suggesting that large numbers of GFP-LC3 labeled autophagosomes had formed and fused with lysosomes during the 10-hour observation period. The accumulation of brightly GFP-LC3 labelled vesicles could be observed as early as 1-2 hours after CQ-treatment (data not shown), but became even more pronounced after 10 hours in inhibitor. Although autophagy can be associated with nutritional deprivation, the gradual accumulation of GFP-LC3 demonstrates that autophagosomes continuously deliver GFP-LC3 for lysosomal degradation, i.e., that autophagy is constitutively active in human epithelial cell lines under nutrient-rich conditions.

To quantify macroautophagy in human B cells and dendritic cells, turnover was visualized of lentivirally delivered GFP-LC3 in EBV-transformed B lymphoblastoid cell lines (LCL) and in monocyte-derived immature and mature DCs. To assess lysosomal turnover of GFP-LC3 in Professional APCs, an EBV-transformed B lymphocyte cell line (B-LCL) stably expressing GFP-LC3 and GFP-LC3-expressing immature and mature DCs (iDC and mDC) were treated with 50 mM chloroquine for 10 hr (+CQ) or were left untreated (no CQ). Cells were fixed, stained with DAPI, and analyzed by confocal microscopy. Scale bars represent 20 mm. Representative fields from one experiment out of two are shown. In all three cell types, GFP-LC3-labeled autophagosomes strongly accumulated after treatment with CQ for 10 hr (FIG. 1B), indicating constitutive autophagosome turnover.

Next, overlap of autophagosomes with MHC class II-loading compartments was investigated in professional APCs, most notably dendritic cells. CD14+ monocytes were tranfected with a lentiviral GFP-LC3 reporter construct, generated immature and mature DCs, and stained them with an MHC class II-specific antibody. Because DCs are constitutively MHC class II-positive, no IFN-g treatment was necessary for these experiments.

MHCclass II compartments of immature DCs frequently contained the autophagosome marker GFP-LC3 after 10 hr of CQ treatment (FIG. 1C, top panel). Colocalization analysis showed that 41% (±7%) of MI-IC class II-labeled compartments were positive for GFP-LC3. In the majority of mature DCs, MHC class II molecules were mainly localized at the cell surface (FIG. 1C, lower panel) and therefore the overlap with autophagosomes was minimal. However, in a subset of cells that still had some intravesicular MHC class II staining, GFP-LC3 was frequently localized within these MIICs after CQ treatment (FIG. 1C, lower panel, white arrow). In the absence of the lysosomal acidification inhibitor CQ, GFP-LC3 was mainly present in the cytosol and only very few GFP-LC3+ vesicles could be observed in both immature and mature DCs (see FIG. 1B). Therefore, no colocalization analysis could be performed for untreated DCs. However, the accumulation of GFP-LC3 in MIICs of CQ-treated immature and mature DCs showed that autophagosomes feed into the MHC class II pathway not only in epithelial cell lines but also in professional APCs, namely dendritic cells.

To show that macroautophagy is required for delivery of MP1-LC3 to MHC class II-loading compartments, MDAMC cells stably expressing MP1-LC3 were transfected with control siRNA (specific for firefly luciferase) or siRNA specific for atg 12. After 36 h, cells were treated with 200 U/ml IFNγ to upregulate MHC class II expression and were cultured for another 36 h. To prevent degradation of MP1-LC3 by lysosomal proteases, cells were treated with 50 μM chloroquine (FIG. 1D; CQ) during the last 6 hours of the culture, where indicated (+CQ). Cells were fixed, stained with MP1- and MHC class II-specific antibodies and DAPI and analyzed by confocal microscopy. Scale bar: 10 μm. Representative fields from one experiment out of two are shown. In control siRNA-treated cells, a substantial fraction of MP1-LC3-containing vesicles can be observed to colocalize with MHC class II compartments, whereas this colocalization is completely abrogated after atg12.

To show that chloroquine treatment induces gradual accumulation of GFP-LC3 in autolysosomes and differs substantially from nutrient starvation, 293 cells stably transfected with a GFP-LC3 reporter construct were left untreated (FIG. 1E, no CQ) or were treated with 50 μM chloroquine (CQ) for 2 or 10 hours. Cells were fixed, stained with DAPI and analyzed by fluorescence microscopy. Inhibition of lysosomal acidification with CQ leads to a gradual accumulation of GFP-LC3-labeled autophagosomes over time. Representative fields from one experiment out of two are shown. In FIG. 1F, MDAMC cells were left untreated (--), cultured in Hanks Balanced Salt Solution (starv.), treated with 50 μM chloroquine (+CQ) or with the protease inhibitors E64 (28 μM), Leupeptin (40 μM) and Pepstatin A (15 μM) (+Prot. inhib.) for 10 hours. Whole cell lysates were run on a 12%® SDS-PAGE gel and LC3-I and II were visualized by anti-LC3 Western blotting. Actin blot demonstrates equal protein loading. While nutrient starvation induces LC3-II only weakly, inhibition of lysosomal proteases by treatment with CQ or the protease inhibitors E64, Leupeptin and Pepstatin A leads to a strong accumulation of LC3-II. One of two experiments is shown. In FIG. 1G, MDAMC cells stably expressing GFP-LC3 were either mock transfected or transfected with siRNA duplexes specific for lamin A/C or ATG12. After 2 days, cells were treated with 50 mM CQ for 6 hr (+CQ) or were left untreated (no CQ), stained with DAPI, and examined in an epifluorescence microscope. One of two experiments is shown. Thus, the accumulation of brightly GFP-LC3-labeled vesicles could already be observed 2 hr after CQ treatment (FIG. 1E), in good agreement with the rapid degradation kinetics of these vesicles. The accumulation of GFP-LC3+ vesicles upon CQ treatment was dependent on macroautophagy, because siRNA-mediated knockdown of ATG12, a gene essential for autophagosome formation completely abrogates accumulation of these vesicles (FIG. 1G).

### Example 2

### Autophagy is a Constitutively Active Process in Human Epithelial Cell Lines and Professional APCs

To extend results obtained with transfected GFP-LC3 to endogenous autophagosomes, the turnover of endogenous LC3 was quantified. This also allowed for extension of the analysis from epithelial cell lines to MHC class II-positive cell types that are more difficult to transfect, such as B cell lines and primary monocytes/dendritic cells. The fact that autophagosome-associated LC3 (called LC3-II) and free cytosolic LC3 (called LC3-I) can be distinguished by their apparent molecular weights in SDS-PAGE gel electrophoresis (16 and 18 lcD, respectively) was used, since it can be quantified separately in anti-LC3 Western blots.

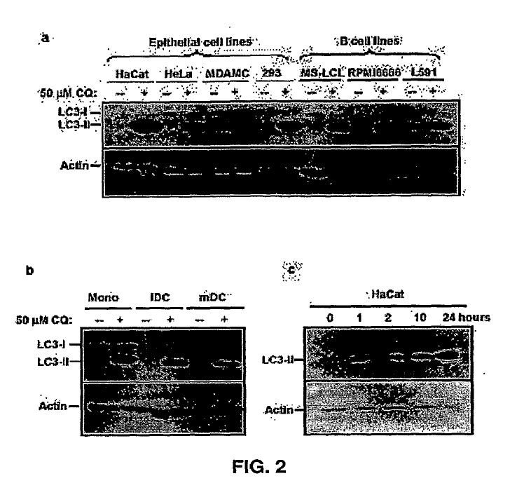

Different human epithelial and B cell lines, primary CD14+ monocytes and monocyte-derived dendritic cells were cultured in the presence or absence of the lysosomal protease inhibitor CQ for 10 hours, and then the accumulation of LC3-II was quantified by immunoblotting. In all cell types examined, autophagosome-associated LC3-II strongly accumulated upon CQ-treatment (FIGS. 2a and b), demonstrating that LC3-II labeled autophagosomes were constitutively degraded in endosomes/lysosomes over the course of 10 hours. As shown in FIG. 2c for the HaCat cell line, cellular LC3-II levels were already increased 1 hour after CQ-treatment and gradually accumulated to after longer treatment times, confirming that autophagosomes are being produced continuously. Density quantification of Western blots revealed that LC3-II accumulated between 5-fold (HaCat and MDAMC cells) and 30-fold (293 cells) after 10 hours of CQ-treatment (data not shown). Taken together, these experiments confirm that autophagy is a constitutively active process in all human cell types analyzed. Furthermore, constitutive autophagy is not restricted to transformed cell lines, but is also a feature of primary cells, as demonstrated for primary monocytes and dendritic cells.

### Example 3

### GFP-LC3Colocalizes with Markers of MHC Class II Loading Compartments in IFNγ-Treated Human Epithelial Cell Lines

To test whether autophagosomes fuse with MHC class II loading compartments (MIICs), confocal microscopy was used to examine whether the autophagosome marker GFP-LC3 would colocalize with markers of MIICs. MIICs have been characterized as conventional late endosomal compartments that in addition to late endosomal/lysosomal markers, such as LAMP-1 and -2, contain for the components for MHC class II loading, namely MHC class II and the peptide-loading chaperone HLA-DM 1.

Analysis was explored with epithelial cells, since they might rely heavily on endogenous

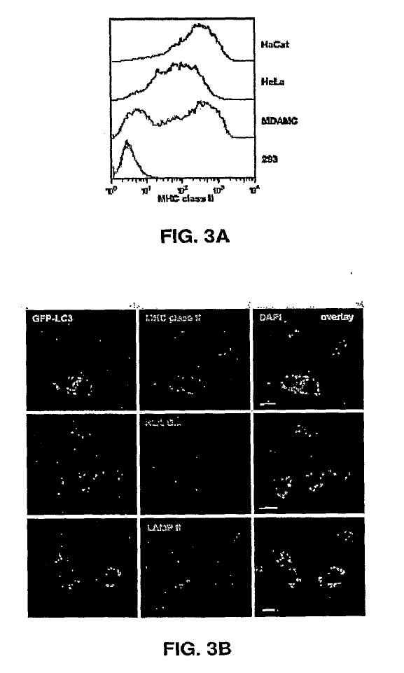

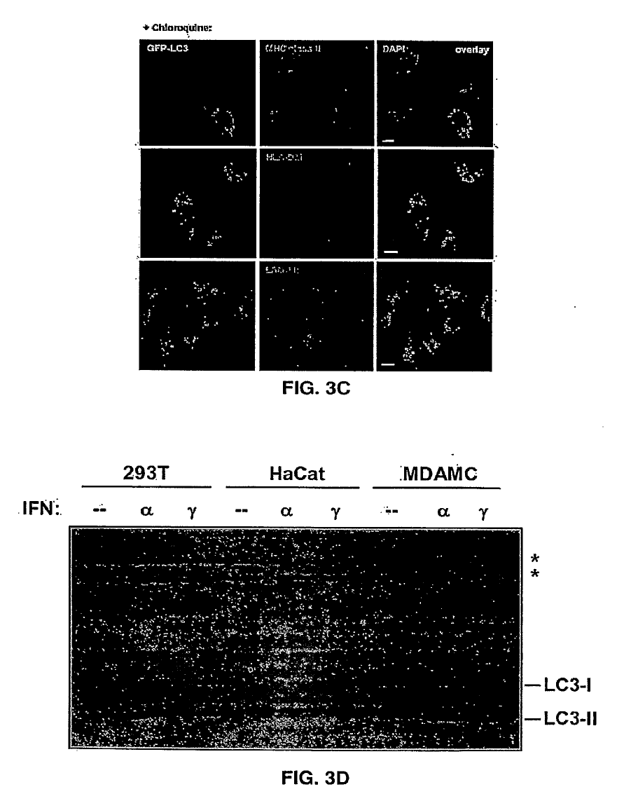

MHC class antigen processing due to their limited endocytic potential. Most of the human epithelial cell lines used, with the exception of 293 cell's, expressed MHC class II molecules after IFNγ treatment (FIG. 3a). For colocalization analysis, cells were treated with IFNγ, transiently transfected with the GFP-LC3 reporter construct, and stained with antibodies specific for the MIIC markers MHC class II, HLA-DM and LAMP-2 in MDAMC (FIG. 3b) and HaCat cells (data not shown). We did not observe the induction of autophagy or changes in autophagosome patterns upon IFNγ treatment of the human cells used in this study (data not shown), but the IFNγ treatment induced the formation of MHC class II positive compartments. As shown in FIG. 3b, these MHC class II positive compartments frequently colocalized with GFP-LC3. When we quantified colocalization of MHC class II and HLA-DM with GFP-LC3 using the LSM510 software's profile tool, which overlays the intensity profiles along a cross section through a cell, we found that among double-positive cells, 58%® of MHC class II+ and 52% of HLA-DM+ compartments were GFP-LC3 positive. Similarly, with the LSM510 software's histogram tool, which quantifies the number of colocalized pixels for pixels above a certain intensity threshold, colocalization with GFP-LC3 was found to be 40% for MHC class II+ and 38%® for HLA-DM+ compartments. To assess the proportion of MIICs, which showed no colocalization with GFP-LC3 due to degradation of the autophagosome marker to protein, we performed the same experiments on chloroquine-treated MDAMC and HaCat cells. Colocalization of GFP-LC3 with MHC class II, HLA-DM and LAMP-2 was more pronounced under these conditions (FIG. 3c and data for HaCat not shown). Although colocalization analysis with the LSM510 histogram tool revealed only a moderate increase (from 40 to 44% for MHC class II and from 38 to 45%® for HLA-DM), analysis with the LSM 510 profile tool showed that after chloroquine treatment, colocalization increased from 58 to 86% for MHC class II+ and from 52 to 71% for HLA-DM+ vesicles. These results demonstrates that the majority of MHC class II loading compartments obtain input from autophagosomes in human epithelial cell lines.

In the human cell lines used in this study, IFNγ treatment did not lead to a detectable upregulation of macroautophagy, as determined by immunoblot (FIG. 3D). Human epithelial cell lines (293T, HaCat and MDAMC) were treated for 24 h with 1000 U/ml recombinant human IFN-α or IFN-γor were left untreated (--). Whole cell lysates were prepared and equal amounts of protein were run on a 12% SDS-PAGE gel. LC3-I and -II were visualized by anti-LC3 Western blotting. The high molecular weight bands marked with an asterisk (*) are proteins that cross-react with the LC3 antiserum and demonstrate equal protein loading. LC3-II levels and hence macroautophagy are not affected by the IFN treatment, although IFN-g treatment could have slightly influenced the macroautophagy activity, in addition to inducing MHC class II-positive compartments.

### Example 4

### Early Endosomes or MHC Class I Loading Compartments Rarely Fuse with GFP-LC3 Labeled Vesicles

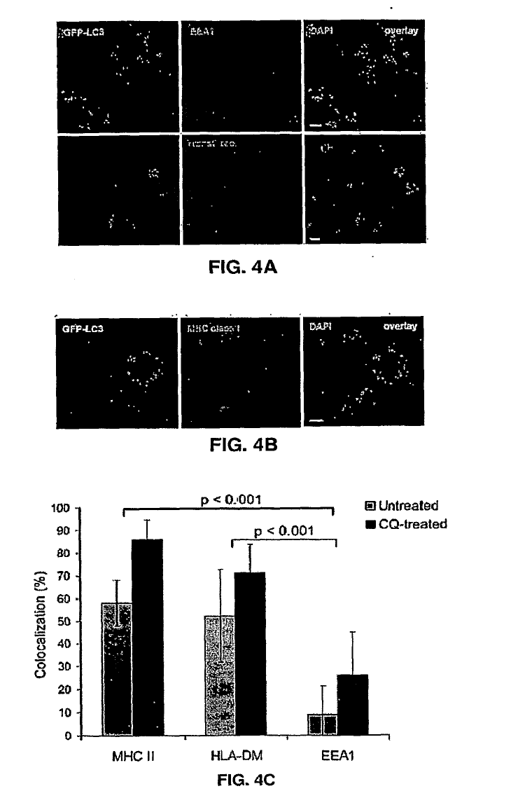

To determine if autophagosomes selectively fuse with MIICs, the overlap of GFP-LC3 with markers of other endocytic compartments was analyzed, specifically early endosomes (positive for early endosomal antigen, EEA1) and recycling endosomes (positive for transferrin receptor, TR). As shown in FIG. 4a for the MDAMC cell line, GFP-LC3 did not colocalize significantly with either EEA1 or transferrin receptor, even in the presence of chloroquine. When colocalization of EEA1 with GFP-LC3 was quantified using the LSM510 software, colocalization was low in untreated MDAMC cells (9% by profile analysis and 7% by histogram analysis), but slightly increased after chloroquine treatment (to 26% and 21%, respectively). The difference between GFP-LC3 colocalization with MHC class II and HLA-DM versus with EEA1 was statistically significant in the presence and absence of CQ (homocedastic student's T test statistics: p<0.001). In HaCat cells, the overlap of GFP-LC3 with EEA1 or transferrin receptor was also minimal (data not shown). Furthermore, in IFNγ treated cells, GFP-LC3 rarely entered early or recycling endosomes (data not shown). Quantitative analysis for colocalization of GFP-LC3 with MHC class II, HLA-DM, and EEA1 in untreated or CQ-treated MDAMC cells is shown in FIG. 4C. Data represent means from 10-15 cells from one representative experiment out of two. Error bars indicate standard deviations. p values from homocedastic, one-tailed Student's t test statistics are shown.

Autophagy has been implicated in the presentation of intracellular antigens on MHC class II, but does not seem to influence MHC class I presentation. To further address this issue, the overlap of GFP-LC3 with MHC class I-molecules was analyzed. As expected, MHC class I was mainly found in perinuclear ER/Golgi regions and on the plasma membrane and did not colocalize with the more peripherally distributed GFP-LC3-positive vesicles (FIG. 4b). Together, the data suggest that autophagosomes mainly fuse with MIICs in MHC class II positive cells, but only rarely with early/recycling endosomes or MHC class I loading compartments.

### Example 5

### GFP-LC3 and MHC Class II Colocalize in Electron-Dense Multivesicular Compartments

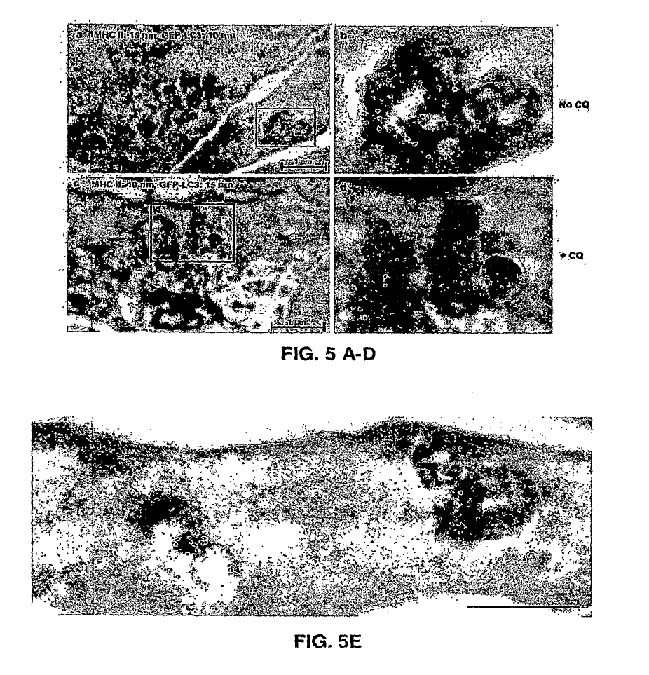

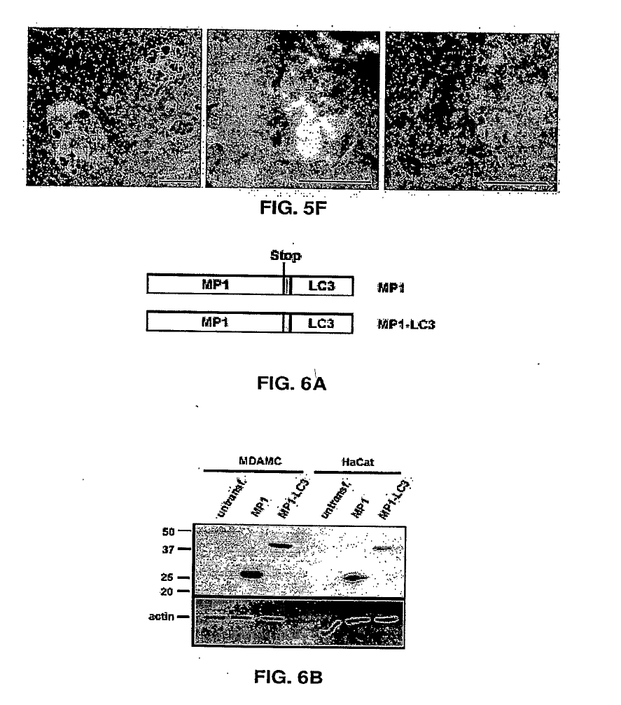

To identify GFP-LC3/MHC class II double-positive compartments by electron microscopy, we prepared ultrathin cryosections of untreated or CQ-treated, stably GFP-LC3 transfected MDAMC cells, stained them with antibodies specific for HLA-DR and GFP, and applied antibodies labeled with 10 and 15 nm protein A-Gold particles. In both untreated and CQ-treated cells, the two antibodies strongly labeled large (1-2 μm), electron-dense, multivesicular compartments, whereas other organelles, such as nuclei and mitochondria, were mostly gold-negative (FIG. 5 a and c). In CQ-treated cells, double-labeled multivesicular compartments were found more frequently than in untreated cells (data not shown). The morphology of the double-labeled compartments was characterized by the presence of electron-dense material, numerous small and sometimes large internal vesicles (FIG. 5 b and d).

Ultrathin cryosections of PFA-fixed MDAMC-GFP-LC3 cells were double-labeled with anti-HLA-DR antiserum/15 nm gold particles and anti-GFP antiserum/10 nm gold particles and analyzed by electron microscopy (FIG. 5E). MHC class II labeling can be seen both on GFP-LC3-positive electron-dense multivesicular compartments and on the plasma membrane. One representative field from one experiment out of three is shown. Scale bar: 1 μm. MDAMC-GFP-LC3 cells were treated with 50 μM CQ for 10 h and ultrathin crysections were double-labeled for MI-IC class H (10 nm gold) and GFP (15 nm gold) and analyzed by electronmicroscopy (FIG. 5F). Double-labeled multivesicular compartments frequently appear expanded and swollen, with a diameter of >1 μm and some empty space. Three representative fields from one experiment out of three are shown. Scale bar: 1 μm.

Other organelles, such as nuclei and mitochondria, were mostly gold negative; however, some GFP-LC3 staining was observed in the cytosol, and MHC class II staining could be seen on the ER, on the Golgi, and at the cell membrane (FIG. 5E). The morphology of the double-labeled compartments was very similar in untreated and CQ-treated cells, but they were found much more frequently in CQ-treated cells (data not shown), and some of them displayed the characteristic swollen phenotype of lysosomal compartments under chloroquine treatment (FIG. 5F). Thus, both GFP-LC3 and MHC class II molecules were often found in close proximity to each other on intraluminal lipid membranes. This suggests that autophagosomes frequently fuse with MHC class II compartments, giving rise to multivesicular compartments that contain both MHC class II molecules and LC3 on internal membranes.

### Example 6

### Cytosolic/Nuclear Antigens are Targeted for Autophagic Degradation by Fusion to Atg81LC3

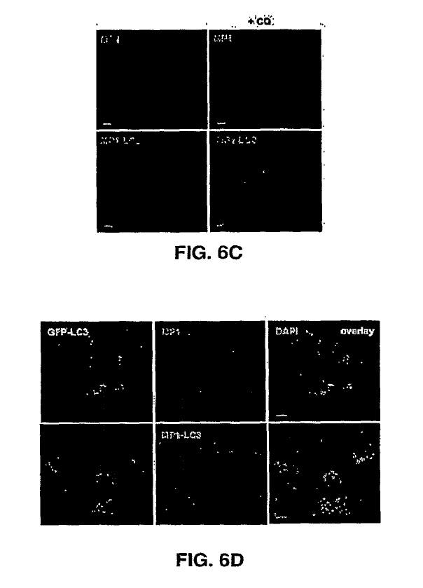

The observation that autophagosomes continuously fuse with MHC class II loading compartments provides a means for cytosolic material to provide peptides for MHC class II loading and thus antigen presentation to CD4+ T cells. To test this hypothesis, it was of interest to determine whether the targeting of a cytosolic antigen for autophagy would lead to enhanced CD4+ T cell recognition. For this purpose, a fusion construct of the Influenza matrix protein 1 with the autophagosome marker protein Atg8/LC3 was generated (FIG. 6a), reasoning that the LC3 portion of such a fusion protein should target the antigen to autophagic membranes and subsequently degradation in MIICs.

A nucleic acid encoding the human LC3 protein was used. The human microtubule associated proteins 1A/1B light chain 3B, Expasy accession: MLP3B_HUMAN (Q9GZQ8) was utilized. The sequence was as follows:

(SEQ ID NO: 1)

atgccgtcgg agaagacctt caagcagcgc cgcaccttcg
aacaaagagt agaagatgtc cgacttattc gagagcagca
tccaaccaaa atcccggtga taatagaacg atacaagggt
gagaagcagc ttcctgttct ggataaaaca aagttccttg
tacctgacca tgtcaacatg agtgagctca tcaagataat
tagaaggcgc ttacagctca atgctaatca ggccttcttc
ctgttggtga acggacacag catggtcagc gtctccacac
caatctcaga ggtgtatgag agtgagaaag atgaagatgg
attcctgtac atggtctatg cctcccagga gacgttcggg
atgaaattgt cagtgtaa.

The nucleic acid encodes a protein with an amino acid sequence as set forth in the EMBL database, accession number AAG23182.

MP1-LC3 fusion proteins were expressed, as described. The nucleic acid sequence encoding the MP1-LC3 fusion proteins, has the following sequence:

(SEQ ID NO: 2)

ATGAGTCTTCTAACCGAGGTCGAAACGTACGTTCTCTCTATCGTCCCGTC
AGGCCCCCTCAAAGCCGAGATCGCACAGAGACTTGAAGATGTCTTTGCAG
GGAAGAACACCGATCTTGAGGTTCTCATGGAATGGCTAAAGACAAGACCA
ATCCTGTCACCTCTGACTAAGGGGATTTTAGGATTTGTGTTCACGCTCAC
CGTGCCCAGTGAGCGGGGACTGCAGCGTAGACGCTTTGTCCAAAATGCTC
TTAATGGGAACGGAGATCCAAATAACATGGACAAAGCAGTTAAACTGTAT
AGGAAGCTTAAGAGGGAGATAACATTCCATGGGGCCAAAGAAATAGCACT
CAGTTATTCTGCTGGTGCACTTGCCAGTTGTATGGGCCTCATATACAACA
GGATG
GGGGCTGTGACCACTGAAGTGGCATTTGGCCTGGTATGCGCAACCTGTGA
ACAGATTGCTGACTCCCAGCATCGGTCTCATAGGCAAATGGTGACAGCAA
CCAATCCACTAATCAGACATGAGAACAGAATGGTTCTAGCCAGCACTACA
GCTAAGGCTATGGAGCAAATGGCTGGATCGAGTGAGCAAGCAGCAGAGGC
CATGGATATTGCTAGTCAGGCCAGGCAAATGGTGCAGGCGATGAGAACCA
TTGGGACTCATCCTAGCTCCAGTGCTGGTCTAAAAGATGATCTTCTTGAA
AATTTGCAGGCCTATCAGAAACGAATGGGGGTGCAGATGCAACGATTCAA
GGACTCGAGCTCAAGCTTCGAATTCACCATGCCGTCGGAGAAGACCTTCA
AGCAGCGCCGCACCTTCGAACAAAGAGTAGAAGATGTCCGACTTATTCGA
GAGCAGCATCCAACCAAAATCCCGGTGATAATAGAACGATACAAGGGTGA
GAAGCAGCTTCCTGTTCTGGATAAAACAAAGTTCCTTGTACCTGACCATG
TCAACATGAGTGAGCTCATCAAGATAATTAGAAGGCGCTTACAGCTCAAT
GCTAATCAGGCCTTCTTCCTGTTGGTGAACGGACACAGCATGGTCAGCGT
CTCCACACCAATCTCAGAGGTGTATGAGAGTGAGAAAGATGAAGATGGAT
TCCTGTACATGGTCTATGCCTCCCAGGAGACGTTCGGGATGAAATTGTCA
GTGTAA.

The plain text corresponds to the MP1 sequence from influenza strain A/WSN/33, similar to MI_IAWIL, the italics represent the linker sequence, and bolded nucleotides represent the LC3 sequence.

The nucleic acid encoded the MP1-LC3 fusion protein, as described, with the following amino acid sequence:

(SEQ ID NO: 3)

MSLLTEVETYVLSIVPSGPLKAEIAQRLEDVFAGKNTDLEVLMEWLKTRP
ILSPLTKGILGFVFTLTVPSERGLQRRRFVQNALNGNGDPNNMDKAVKLY
RKLKREITFHGAKEIALSYSAGALASCMGLIYNRMGAVTTEVAFGLVCAT
CEQIADSQHRSHRQMVTATNPLIRHENRMVLASTTAKAMEQMAGSSEQAA
EAMDIASQARQMVQAMRTIGTHPSSSAGLKDDLLENLQAYQKRMGVQMQR
FKDSSSSFEFTMPSEKTFKQRRTFEQRVEDVRLIREQHPTKIPVIIERYK
GEKQLPVLDKTKFLVPDHVNMSELIKIIRRRLQLNANQAFFLLVNGHSMV
SVSTPISEVYESEKDEDGFLYMVYASQETFGMKLSV

The MP1-LC3 fusion protein or the MN control protein was then stably expressed in the human epithelial cell lines HaCat and MDAMC (FIG. 6a). Western blot analysis showed that the antigens were expressed in both cell lines and had the expected molecular weights (MP1: 28 kD; MP1-LC3: 43 kD) (FIG. 6b). Notably, the MP1-LC3 fusion protein was present at slightly lower levels than MP1 in both cell lines. In order to assess the targeting behavior of the fusion protein, their localization by immunocytochemistry was investigated. As expected, wild-type MP1 was localized in the cytosol and nucleus, with some cytosolic and nuclear speckles, and this pattern did not change dramatically after CQ-treatment (HaCat: FIG. 6c; MDAMC: data not shown). In contrast, the MP1-LC3 fusion protein showed punctate cytosolic staining, which was further enhanced upon inhibition of lysosomal proteolysis with CQ (FIG. 6c). To test whether MP1-LC3 positive cytosolic punctae were autophagosomes, GFP-LC3 and MP1-LC3 were coexpressed in HaCat and MDAMC cell lines and their colocalization analyzed by confocal microscopy. In both cell lines, GFP-LC3 and MP1-LC3 fusion protein accumulated in the same cytosolic vesicles after CQ-treatment, whereas native MP1 did not significantly colocalize with GFP-LC3-labeled compartments (MDAMC: FIG. 6d; HaCat: data not shown). This demonstrates that the LC3 tag indeed targets cytosolic antigens to autophagosomes and for subsequent degradation by lysosomal proteases.

### Example 7

Targeting of Antigens for Autophagic Degradation Leads to Enhanced CD4+ T Cell Recognition

To test the hypothesis that targeting of cytosolic/nuclear antigens for autophagic degradation via LC3 fusion leads to enhanced MHC class II presentation, the recognition of MP1-versus MP1-LC3-expressing target cells by MP1-specific CD4+ T cell clones was analyzed. For this purpose, MP1-specific CD4+ T cell clones were generated (FIG. 7) from a donor that was HLA-DR and -DQ matched to the HaCat cell line, so that IFNγ-treated HaCat cells could be used as target cells. The CD4+ T cell clones were homogenously CD4 positive and recognized the MP162-72 peptide sequence of overlapping peptides in a MP1 peptide library. To analyze the effect of the LC3 fusion on MHC class I presentation, we also generated an MP158-66-specific, HLA-A2-restricted CD8+ T cell clone from the same donor (FIG. 7), which then could be tested for recognition of HLA-A2 positive MDAMC target cells.

FIGS. 7 A-C demonstrate the characterization of influenza MP1 specific CD4+ and CD8+. T cell clones. In FIG. 7a, CD4 and CD8 expression of the clones was analyzed by flow cytometry. Clones 9.26, 11.46 and 10.9 were homogenously CD4+CD8− and clone 9.2 homogenously CD8+CD4−. In FIG. 7B, their recognition of Influenza MP1 peptides was tested by IFNγ ELISPOT assays. The MP1 peptide library was divided in 6 subpools covering MP1 amino acid positions 1-51 (pool I), 41-88 (pool II), 78-128 (pool III), 118-163 (pool IV), 152-203 (pool V) and 193-252 (pool VI). Clones 9.2, 9.26 and 10.9 responded specifically to pool II and clone 11.46 to pool III. In addition, the CD8+ T cell clone 9.2, but not the CD4+ T cell clones, recognized the HLAA2 restricted MP1 epitope 58-66. Error bars indicate standard deviations. In FIG. 7c, MP1-specific CD4+ T cell clones were tested for recognition of individual peptides covering MP1 amino acid sequence 29-128, including all peptides of MP1 pools II and TIT. Clones 9.26 and 10.9 specifically recognized peptide epitope MP162-72 and clone 11.46 was specific for epitope MP1103-113. Error bars indicate standard deviations.

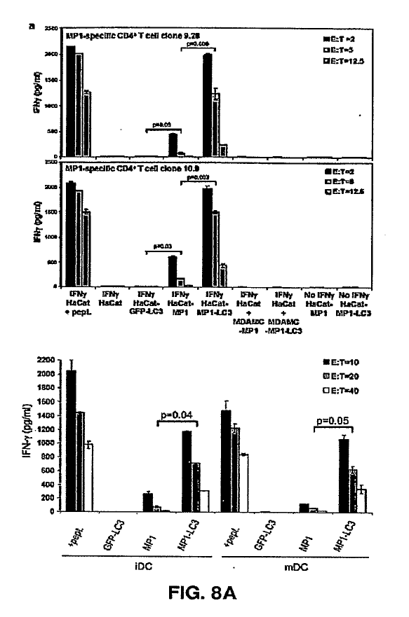

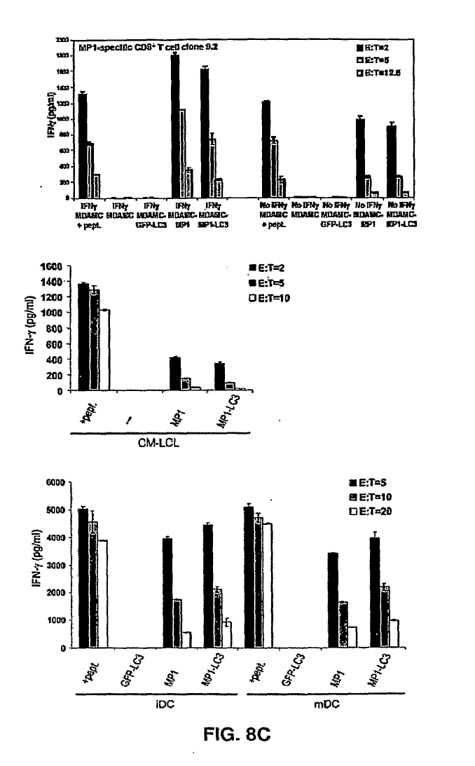

To assess how well the two different forms of MP1 could be presented on MHC class II, we measured IFNγ secretion of two MP1-specific CD4+ T cell clones in response to MP1 or MP1-LC3-expressing HaCat target cells. IFNγ ELISA assays showed that the response of both CD4+ T cell clones (clone 9.26 and 10.9, FIG. 8a, upper two panels, respectively) was strongly increased by the LC3 fusion. While at the lowest ratio of T cell clone to cell line targets (effector to target or E:T ratio of 2) MP1-LC3 elicited only 3-4 fold higher IFNγ production (homocedastic student's T test statistics: p<0.001), the difference in IFNγ secretion was especially pronounced at higher E:T ratios (5 and 12.5), when the target cells and thus MHC class II-peptide complexes became limiting. At these E:T ratios, the IFNγ secretion by the CD4+ T cell clones was increased between 9-17 fold (homocedastic student's T test statistics: p<0.003) in response to MP1-LC3 compared to MP1 (paired student's T test statistics across all E:T ratios: p<0.007). Untransfected and GFP-LC3 transfected HaCat cells were not recognized above background (FIG. 8a, columns 2 and 3). While the IFNγ response to MP1 transfectants never exceeded 30% of the amount secreted upon recognition of the peptide pulsed HaCat positive control, MP1-LC3 was able to stimulate up to 95% of the maximal D4+ T cell recognition achieved with peptide pulsed targets (FIG. 8a, two upper panels, columns 1, 4 and 5). Mixing experiments demonstrated that MHC class II presentation of MP1 and MP1-LC3 was indeed due to endogenous processing, since the mixing of HLA-matched HaCat cells with mismatched MP1- or MP1-LC3 expressing MDAMC cells did not stimulate any T cell responses (FIG. 8a, columns 6 and 7). Furthermore, when MHC class II was not induced by IFNγ, MP1- and MP1-LC3-expressing HaCat cells were unable to stimulate CD4+ T cell responses (FIG. 8a, two upper panels, columns 8 and 9), confirming that the presentation was MHC class II-restricted. FIG. 8a, lower panel, shows results for clone 11.46 for immature/mature DCs at effector to target (E:T) ratios of 10, 20 and 40. In addition, MHC class II surface staining showed that MHC class II was upregulated similarly in response to IFNγ in MP1 and MP1-LC3-expressing target cells (FIG. 8b), demonstrating that the enhanced recognition of MP1-LC3 was not due to an enhanced MHC class expression level.

To assess the effect of the LC3 fusion on MHC class I presentation, we analyzed the IFNγ response of an MP1-specific CD8+ T cell clone to MP1- and MP1-LC3-expressing MDAMC target cells. IFNγ ELISA showed that for all three E:T ratios, similar amounts of IFNγ were secreted by CD8+ T cells in response to MP1- and MP1-LC3 expressing targets (FIG. 7c), suggesting that the LC3 fusion does not impair MHC class I presentation and both constructs probably give rise to similar amounts of defective ribosomal products (DRiPs), which are then efficiently processed for CD8+ T cell recognition. This was observed for both IFNγ treated target cells (FIG. 8c, upper panel, columns 1-5) and untreated target cells (FIG. 8c, upper panel, columns 7-11), although MHC class I presentation seemed to be slightly enhanced by the IFNγ treatment, which is consistent with an enhanced MHC class I processing machinery. Taken together, the data suggest that targeting of cytosolic antigens for autophagic degradation via LC3 fusion can strongly increase CD4+ T cell recognition, without impairing CD8+ T cell recognition. FIG. 8c, middle and lower panels show corresponding results with CM-LCL and immature/mature DCs, respectively.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

What is claimed is:

1. A nucleic acid encoding a peptide or protein of interest fused in frame to a nucleic acid encoding the autophagosomal LC3 protein, or a functional fragment thereof, wherein said peptide or protein of interest is poorly or not presented efficiently on a major histocompatibility complex (MHC) class II molecule.

2. The nucleic acid of claim 1, wherein said nucleic acid has a sequence homologous to, or corresponding to SEQ ID NO: 1.

3. The nucleic acid of claim 1, wherein said peptide or protein of interest is virally encoded.

4. The nucleic acid of claim 1, wherein said peptide or protein of interest is encoded by the influenza virus.

5. The nucleic acid of claim 4, wherein said peptide or protein of interest is a matrix protein.

6. The nucleic acid of claim 5, wherein said nucleic acid has a sequence homologous to, or corresponding to SEQ ID NO: 2.

7. A vector or cell comprising the nucleic acid of claim 1.

8. A cell comprising the vector of claim 7.

9. A method for stimulating or enhancing presentation of a peptide or protein of interest in the context of a major histocompatibility (MHC) class II molecule, the method comprising contacting a cell capable of expressing a major histocompatibility complex (MHC) class II molecule with a nucleic acid encoding a peptide or protein of interest fused in frame to a nucleic acid encoding an autophagosomal targeting protein, or a functional fragment thereof, whereby said autophagosomal targeting protein or a functional fragment thereof targets said peptide or protein of interest to an autophagosome, and said peptide, or a fragment of said protein of interest is displayed on the surface of said cell in the context of a major histocompatibility complex (MHC) class II molecule.

10. The method of claim 9, wherein said cell is contacted with a vector comprising said nucleic acid.

11. The method of claim 9, wherein said autophagosomal targeting protein is an LC3 protein.

12. The method of claim 11, wherein said nucleic acid comprises a sequence homologous to, or corresponding to SEQ ID NO: 1.

13. The method of claim 9, wherein said cell is healthy or diseased and said peptide or protein of interest is associated with the disease.

14. The method of claim 13, wherein said cell is infected.

15. The method of claim 14, wherein said cell is infected with a virus.

16. The method of claim 15, wherein said virus is influenza or HIV.

17. The nucleic acid of claim 14, wherein said peptide or protein of interest is virally encoded.

18. The method of claim 14, wherein said peptide or protein of interest is encoded by the influenza virus.

19. The method of claim 18, wherein said peptide or protein of interest is a matrix protein.

20. The method of claim 19, wherein said nucleic acid has a sequence homologous to, or corresponding to SEQ ID NO: 2.

21. The method of claim 14, wherein said cell is infected with a bacterium.

22. The method of claim 21, wherein said bacteria is a mycobacterium.

23. The method of claim 13, wherein said cell is neoplastic or preneoplastic.

24. The method of claim 9, wherein said cell is a monocyte, macrophage, dendritic cell, B cell or epithelial cell.

25. The method of claim 24, wherein said cell is contacted with interferon-γ.

26. A method for stimulating or enhancing an immune response in a subject, the method comprising contacting a cell capable of expressing a major histocompatibility complex (MHC) class II molecule in said subject with a nucleic acid encoding a peptide or protein of interest fused in frame to a nucleic acid encoding an autophagosomal targeting protein, or a functional fragment thereof, whereby said autophagosomal targeting protein or functional fragment thereof targets said peptide or protein of interest to an autophagosome, and said peptide, or a fragment of said protein of interest is displayed on the surface of said cell in the context of a major histocompatibility complex (MHC) class II molecule, thereby stimulating or enhancing an immune response thereto.

27. The method of claim 26, wherein said cell is contacted with a vector comprising said nucleic acid.

28. The method of claim 26, wherein said autophagosomal targeting protein is an LC3 protein.

29. The method of claim 28, wherein said nucleic acid comprises a sequence homologous to, or corresponding to SEQ ID NO: 1.

30. The method of claim 26, wherein said cell is diseased or healthy and said peptide or protein of interest is associated with the disease.

31. The method of claim 30, wherein said cell is infected.

32. The method of claim 31, wherein said cell is infected with a virus.

33. The method of claim 32, wherein said virus is influenza or HIV.

34. The nucleic acid of claim 32, wherein said peptide or protein of interest is virally encoded.

35. The method of claim 34, wherein said peptide or protein of interest is encoded by the influenza virus.

36. The method of claim 35, wherein said peptide or protein of interest is a matrix protein.

37. The method of claim 32, wherein said nucleic acid has a sequence homologous to, or corresponding to SEQ ID NO: 2.

38. The method of claim 31, wherein said cell is infected with a bacterium.

39. The method of claim 38, wherein said bacteria is a mycobacterium.

40. The method of claim 30, wherein said cell is neoplastic or preneoplastic.

41. The method of claim 26, wherein said cell is a monocyte, macrophage, dendritic cell, B cell or epithelial cell.

42. The method of claim 41, wherein said cell is contacted with interferon-γ.

43. The method of claim 26, wherein said cell is contacted indirectly with said nucleic acid or vector comprising the same.

44. The method of claim 43, wherein said nucleic acid or vector comprising the same is administered intravenously to said subject.

45. The method of claim 44, wherein said subject is administered a composition comprising said nucleic acid or vector comprising the same.

46. The method of claim 45, wherein said composition is administered repeatedly, over a course of time.

47. The method of claim 44, wherein said composition comprises a neoplastic cell isolated from said subject.

48. The method of claim 26, wherein said cell is contacted ex vivo with said nucleic acid or vector comprising the same.

49. The method of claim 48, wherein said subject has preneoplastic or hyperplastic cells or tissue.

50. The method of claim 48, wherein said subject is predisposed to neoplasia.

51. A composition comprising a cell capable of expressing the major histocompatibility complex (MHC) class II protein, and a nucleic acid encoding a peptide or protein of interest fused in frame to a nucleic acid encoding the autophagosomal LC3 protein, or functional fragment thereof, or a vector comprising the same.

52. The composition of claim 51, wherein said nucleic acid comprises a sequence homologous to, or corresponding to SEQ ID NO: 1.

53. The composition of claim 51, wherein said peptide or protein of interest is virally encoded.

54. The composition of claim 53, wherein said peptide or protein of interest is encoded by the influenza virus.

55. The composition of claim 54, wherein said peptide or protein of interest is a matrix protein.

56. The composition of claim 55, wherein said nucleic acid has a sequence homologous to, or corresponding to SEQ ID NO: 2.

57. The composition of claim 51, wherein said cell is a hyperplastic, preneoplastic or neoplastic cell.

58. The composition of claim 51, wherein said cell is a healthy cell and said peptide or protein of interest is associated with a cancer or an infection.

59. The composition of claim 51, further comprising CD4+ T cells.

60. The composition of claim 59, wherein said CD4+ T cells are autologous, syngeneic or allogeneic with respect to said cell expressing the major histocompatibility complex (MHC) class II protein.

61. The composition of claim 51, further comprising a cytokine.

62. The composition of claim 51, wherein said cytokine if interferon-γ.

63. The composition of claim 51, wherein said composition is formulated for intravenous administration.

64. A method for downmodulating, suppressing or tolerizing an immune response in a subject to a peptide or protein of interest, the method comprising contacting immature dendritic cells with a nucleic acid encoding a peptide or protein of interest fused in frame to a nucleic acid encoding an autophagosomal targeting protein, or a functional fragment thereof, whereby said autophagosomal targeting protein or functional fragment thereof targets said peptide or protein of interest to an autophagosome, and said peptide, or a fragment of said protein of interest is displayed on the surface of said immature dendritic cell in the context of a major histocompatibility complex (MHC) class II molecule.

65. The method of claim 64, wherein said cell is contacted in vivo or ex vivo with a vector comprising said nucleic acid.

66. The method of claim 64, wherein said autophagosomal targeting protein is an LC3 protein.

67. The method of claim 66, wherein said nucleic acid comprises a sequence homologous to, or corresponding to SEQ ID NO: 1.

68. The method of claim 64 wherein the downmodulating, suppressing or tolerizing an immune response is to prevent or diminish transplant rejection or graft-vs.-host disease in the subject.

69. The method of claim 68 wherein the peptide or protein of interest is a graft antigen or a host antigen.

70. The method of claim 64 wherein the downmodulating, suppressing or tolerizing an immune response is to treat an autoimmune disease in the subject.

71. The method of claim 70 wherein the peptide or protein of interest is a self antigen.
